(19) United States
(12) Patent Application Publication
(10) Pub. No.: US 2024/0074740 A1
(43) Pub. Date: Mar. 7, 2024

(54) SYSTEMS, METHODS, AND COMPUTER PROGRAM PRODUCTS FOR INTEGRATING MENSTRUAL CYCLE DATA AND PROVIDING CUSTOMIZED FEMININE WELLNESS INFORMATION

(71) Applicant: 28 Wellness, Inc., Miami, FL (US)

(72) Inventors: Brittany Hugoboom, Miami, FL (US); Gabriel Hugoboom, Miami, FL (US)

(73) Assignee: 28 Wellness, Inc., Miami, FL (US)

(21) Appl. No.: 18/236,077

(22) Filed: Aug. 21, 2023

Related U.S. Application Data
(60) Provisional application No. 63/453,330, filed on Mar. 20, 2023.
(60) Provisional application No. 63/399,696, filed on Aug. 21, 2022.

(51) Int. Cl. A61B 10/00; G16H 20/30; G16H 20/60

(52) U.S. Cl. CPC A61B 10/0012; G16H 20/30; G16H 20/60

(57) ABSTRACT

A method for enabling users to input menstrual cycle data and receive customized wellness information, including streaming workouts, nutrition profiles, recipes, and physiological data such as science-based “horoscope-like” insights into the user's emotions, tailored to a user's expected physiology within a menstrual cycle phase is provided. The method includes receiving, from a device, menstrual cycle data for a user, wherein the menstrual cycle data includes one or more of a first date of the user's last menstrual cycle, a cycle length of the user's menstrual cycle and/or a length of the user's period, determining menstrual cycle phase data using the menstrual cycle data, determining feminine wellness information using the menstrual cycle phase data, wherein the feminine wellness information includes personalized interactive content, and transmitting, to the device, the menstrual cycle phase data and the feminine wellness information including the personalized interactive content.

### CROSS-REFERENCE TO RELATED APPLICATIONS

This application claims priority benefit under 35 U.S.C. § 119(e) of U.S. Provisional Patent Application No. 63/453,330, filed Mar. 20, 2023, and of U.S. Provisional Patent Application No. 63/399,696, filed Aug. 21, 2022, both entitled “Systems, Methods, And Computer Program Products For Integrating Menstrual Cycle Data And Providing Customized Feminine Wellness Information.” The entire contents of both applications are incorporated herein by reference in their entirety.

### TECHNICAL FIELD

Disclosed are embodiments related to systems, methods, and non-transitory computer program products for enabling users to input menstrual cycle data and receive customized wellness information, including streaming workouts, nutrition profiles, recipes, and physiological data, such as science-based “horoscope-like” insights into the user's emotions, tailored to a user's expected physiology within a menstrual cycle phase.

### BACKGROUND

Many fitness methods are not actually good for women's bodies. Overly strenuous workouts create major hormone imbalances and bad form that leads to long-term swelling and injuries. A majority of women are in the dark about their own body and many do not know there are four phases to their cycle. From an early age, some women are put on the birth control pill, which can mask their issues.

Every woman has four phases within their menstrual cycle: Menstrual, Follicular, Ovulation, and Luteal. Each phase brings different changes to the mood and body.

During the menstrual phase, a woman's period has arrived. As the body sheds the uterine lining, hormone and energy levels are at their lowest.

During the follicular phase, a new cycle has begun and a woman's body is replenishing. Estrogen and testosterone levels rise, making this an optimal phase for muscle growth.

During the ovulatory phase, an egg is released. A woman's fertile window has arrived. A woman may experience positive mood, high energy, and strong sex drive.

During the luteal phase, a woman's body starts to wind down in preparation for menstruation. Progesterone, the dominant hormone, increases, bringing symptoms of PMS.

Conventional exercise programs do not take into account these four phases within a woman's menstrual cycle. Conventional exercise programs do not, based on a woman's menstrual cycle, change the intensity, movements, and types of exercises during each phase to maintain a healthy balance in weight, hormone levels, and overall mood.

### SUMMARY

Disclosed embodiments provide systems, methods, and non-transitory computer program products for customized streaming workouts, nutrition profiles, recipes, and physiological data, such as science-based “horoscope-like” insights into your emotions, all with the purpose of optimizing women's health by working with the changing phases of their natural cycle, not against them. Disclosed embodiments include technology that determines where the user is in their menstrual cycle and caters their experience to their physical and emotional needs.

The disclosed embodiments leverage a woman's fluctuating energy and hormone levels to lengthen, strengthen, stretch, and tone the body throughout the month to achieve a natural, feminine physique. The disclosed embodiments provide a rejuvenating, stability-based, rehabilitation approach to exercising the female body using minimal to no equipment. This approach reduces swelling in the muscles, emphasizes proper form over intensity, and strengthens without bulking for an elongated, soft, feminine look. It is designed around the female cycle, promoting healthy energy levels and key hormone balance. Further, it works with every body type.

Disclosed embodiments provide for users to receive customized wellness information based on menstrual cycle data input. Wellness information can, for example, include streaming workouts, nutrition profiles, recipes, and physiological data, such as science-based “horoscope-like” insights into the user's emotions, tailored to a user's expected physiology within a menstrual cycle phase. Disclosed embodiments provide for tracking a user's menstrual cycle phase and for providing users with daily updated wellness information without the user having to input updated menstrual cycle data.

In a first aspect, embodiments directed to systems, methods, and computer program products for providing feminine wellness information to one or more users are provided. The computer-implemented method for providing feminine wellness information to one or more users may include receiving, from a device, menstrual cycle data for a user, wherein the menstrual cycle data includes one or more of a first date of the user's last menstrual cycle, a cycle length of the user's menstrual cycle, and/or a length of the user's period; determining menstrual cycle phase data using the menstrual cycle data; determining feminine wellness information using the menstrual cycle phase data, wherein the feminine wellness information includes personalized interactive content; and transmitting, to the device, the menstrual cycle phase data and the feminine wellness information including the personalized interactive content. The feminine wellness information may include nutrition profiles physiological data, such as science-based “horoscope-like” insights into a user's emotions. The fitness routine recommendation may be a video of a fitness routine.

In some embodiments, the feminine wellness information includes one or more of a fitness routine recommendation, a nutrition profile, a recipe, and/or physiological data.

In some embodiments, determining feminine wellness information using the menstrual cycle phase data includes using a cycle syncing content recommendation engine algorithm. In some embodiments, inputs to the cycle syncing content recommendation engine algorithm include one or more of a start date of the user's menstrual cycle, a cycle length of the user's menstrual cycle, and/or a length of the user's period; and outputs from the cycle syncing content recommendation engine algorithm include one or more of curated content targeted specifically to where the user is in the user's menstrual cycle.

In some embodiments, determining menstrual cycle phase data using the menstrual cycle data includes using information collected from a wearable device worn by the user, wherein the collected information includes one or more of the user's body and/or skin temperature, heart rate, physical activity, sleep patterns, hormone levels, menstrual symptoms, and/or mood.

In some embodiments, determining feminine wellness information using the menstrual cycle phase data includes using an artificial intelligence (AI)/machine learning (ML) model for providing cycle syncing content recommendations.

In some embodiments, determining feminine wellness information using the menstrual cycle phase data includes using a generative artificial intelligence (GAI) module to generate personalized feminine wellness information. In some embodiments, the GAI generated personalized feminine wellness information includes personalized interactive content including one or more of personalized fitness routines, exercise routines, and/or video workouts

In a second aspect, a computer-implemented method for providing feminine wellness information to one or more users may include receiving, from a device, menstrual cycle data for a user, wherein the menstrual cycle data includes a first date of a last menstrual cycle; receiving, from a device, a first current date; calculating a first number of days since the first date of the last menstrual cycle using the first current date and the menstrual cycle data; determining a first menstrual cycle phase data using the first number of days since the first date of the last menstrual cycle; determining a first feminine wellness information using the first menstrual cycle phase data, wherein the first feminine wellness information includes first personalized interactive content; and transmitting, to the device, the first menstrual cycle phase data and the first feminine wellness information including the first personalized interactive content.

The method may further include receiving, from the device, a second current date; calculating a second number of days since the first date of the last menstrual cycle using the second current date and the menstrual cycle data; determining a second menstrual cycle phase data using the second number of days since the second date of the last menstrual cycle; determining a second feminine wellness information using the second menstrual cycle phase data, wherein the second feminine wellness information includes second personalized interactive content; and transmitting, to the device, the second menstrual cycle phase data and the second feminine wellness information including the second personalized interactive content.

In a third aspect, a computer-implemented method for receiving feminine wellness information for a user may include inputting, in a device, menstrual cycle data, wherein the menstrual cycle data includes one or more of a first date of the user's last menstrual cycle, a cycle length of the user's menstrual cycle, and/or a length of the user's period; transmitting, from the device, the inputted menstrual cycle data; receiving, in the device, the menstrual cycle phase data and the feminine wellness information including personalized interactive content; and displaying, on the device, the received menstrual cycle phase data and the feminine wellness information including the personalized interactive content.

In a fourth aspect, a device comprising a processing circuitry and a memory storing program code that, when executed by the processing circuitry, causes the device to carry to perform the method of any one of disclosed embodiments is provided.

In a fifth aspect, computer program product comprising a non-transitory computer readable medium storing a computer program comprising instructions which, when executed on at least one processor, cause the at least one processor to carry out the method of any one of disclosed embodiments is provided.

### BRIEF DESCRIPTION OF THE DRAWINGS

The accompanying drawings, which are incorporated herein and form part of the specification, illustrate various embodiments of the present disclosure and, together with the description, further serve to explain the principles of the disclosure and to enable a person skilled in the pertinent art to make and use the embodiments disclosed herein. In the drawings, like reference numbers indicate identical or functionally similar elements.

FIG. 1 illustrates an architecture of a communications system according to exemplary embodiments.

FIG. 2 illustrates a block diagram of a user device according to exemplary embodiments.

FIG. 3 illustrates a block diagram of a wearable device according to exemplary embodiments.

FIG. 4 illustrates a block diagram of a remote device according to exemplary embodiments.

FIG. 5 illustrates a flow chart for a process according to exemplary embodiments.

FIG. 6 illustrates a block diagram of an exemplary architecture according to exemplary embodiments.

FIG. 7 illustrates a flow chart for a process according to exemplary embodiments.

FIG. 8 illustrates a block diagram of an exemplary architecture for client-service interaction according to exemplary embodiments.

FIG. 9 illustrates an exemplary screenshot from an application displayed on a user device according to exemplary embodiments.

FIG. 10 illustrates an exemplary screenshot from an application displayed on a user device according to exemplary embodiments.

FIG. 11 illustrates an exemplary screenshot from an application displayed on a user device according to exemplary embodiments.

FIG. 12 illustrates an exemplary screenshot from an application displayed on a user device according to exemplary embodiments.

FIG. 13 illustrates an exemplary screenshot from an application displayed on a user device according to exemplary embodiments.

FIG. 14 illustrates an exemplary screenshot from an application displayed on a user device according to exemplary embodiments.

FIG. 15 illustrates an exemplary screenshot from an application displayed on a user device according to exemplary embodiments.

FIG. 16 illustrates an exemplary screenshot from an application displayed on a user device according to exemplary embodiments.

FIG. 17 illustrates an exemplary screenshot from an application displayed on a user device according to exemplary embodiments.

FIG. 18 illustrates an exemplary screenshot from an application displayed on a user device according to exemplary embodiments.

FIG. 19 illustrates an exemplary screenshot from an application displayed on a user device according to exemplary embodiments.

FIG. 20 illustrates an exemplary screenshot from an application displayed on a user device according to exemplary embodiments.

FIG. 21 illustrates an exemplary screenshot from an application displayed on a user device according to exemplary embodiments.

FIG. 22 illustrates an exemplary screenshot from an application displayed on a user device according to exemplary embodiments.

FIG. 23 illustrates an exemplary screenshot from an application displayed on a user device according to exemplary embodiments.

FIG. 24 illustrates an exemplary screenshot from an application displayed on a user device according to exemplary embodiments.

FIG. 25 illustrates an exemplary screenshot from an application displayed on a user device according to exemplary embodiments.

FIG. 26 illustrates an exemplary screenshot from an application displayed on a user device according to exemplary embodiments.

FIG. 27 illustrates an exemplary screenshot from an application displayed on a user device according to exemplary embodiments.

FIG. 28 illustrates an exemplary screenshot from an application displayed on a user device according to exemplary embodiments.

FIG. 29 illustrates an exemplary screenshot from an application displayed on a user device according to exemplary embodiments.

### DETAILED DESCRIPTION

Some of the embodiments contemplated herein will now be described more fully with reference to the accompanying drawings. Embodiments are provided by way of example to convey the scope of the subject matter to those skilled in the art. Additional information may also be found in the document(s) provided in the Appendix.

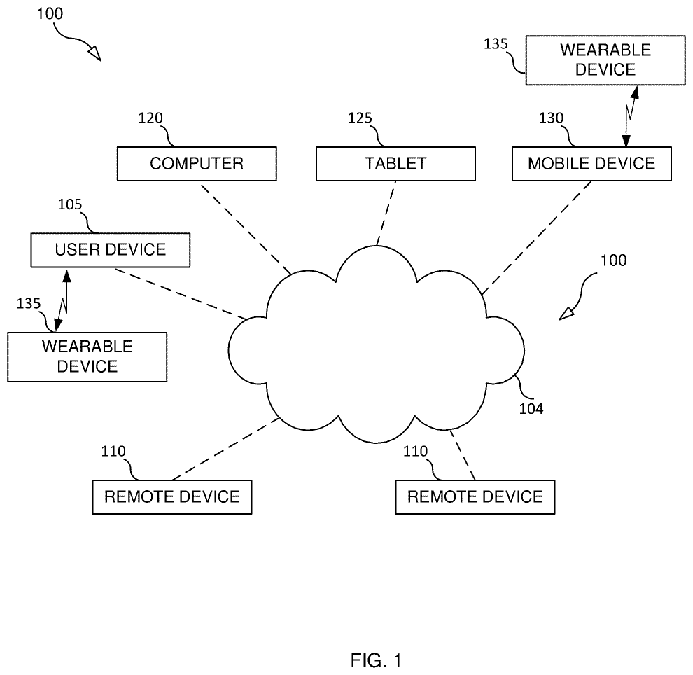

Referring now to FIG. 1, an exemplary architecture of a communication system in accordance with some embodiments is illustrated. System 100 includes at least one remote device or node 110 that is configured to communicate with one or more user devices or nodes 105 through a communications network 104 (e.g., the internet). Examples of user devices and nodes include a computer 120 (e.g., laptop or desktop), a tablet 125 (e.g., an iPad), and a mobile device 130 (e.g., a smartphone, such as, for an example, an iPhone). An example of a remote device or node 110 includes a server, peer, or like device or node.

The system 100 may also include one or more wireless wearable devices 135. Examples of wearable devices include a watch (e.g., Apple Watch, Garmin), a fitness tracker (e.g., FitBit, Whoop), and a ring (e.g., Oura Ring, Movano Health Evie Ring).

References to devices or nodes herein should be understood such that a device or node may be a physical node or a function or logical entity of any kind, e.g. a software entity implemented in a data center or a cloud, e.g. using one or more virtual machines.

The systems, methods and computer program products of the disclosed embodiments can, for example, be deployed as a user/client-server implementation, as an application service provider (ASP) model, as a standalone application running on a user device 105, in a peer-to-peer (P2P) environment, in a cloud-based environment, and the like.

The user device 105 can be configured to communicate with one or more remote devices 110 via the network 104. Remote devices 110 are configured to generate, maintain, and host the computer program product in some of the disclosed embodiments. The remote devices 110 can generate, maintain and host web pages (e.g., HTML documents) in accordance with disclosed embodiments. The remote devices 110 can include services associated with rendering dynamic web pages, such as data storage services, security services, etc. Accordingly, remote devices 110 can include a conventional hardware arrangement and can be outfitted with software and/or firmware for performing web server functions for performing aspects of the disclosed embodiments, such as, for example, javascript/jquery, HTML5, CSS2/3, and facilities for SSL, MySQL, PHP, SOAP, etc.

Remote devices 110 may be coupled with a data storage facility, which may include one or more local or remote memory systems or units, and can include one or more databases and/or file systems for storing data, media, graphics, HTML documents, XML documents, etc.

Remote devices 110 can be configured to include an admin function, which enables an administrator to perform system-related functions. The system-related functions can include maintaining user records, performing upgrades on the software and topic content, and the moderation of tasks.

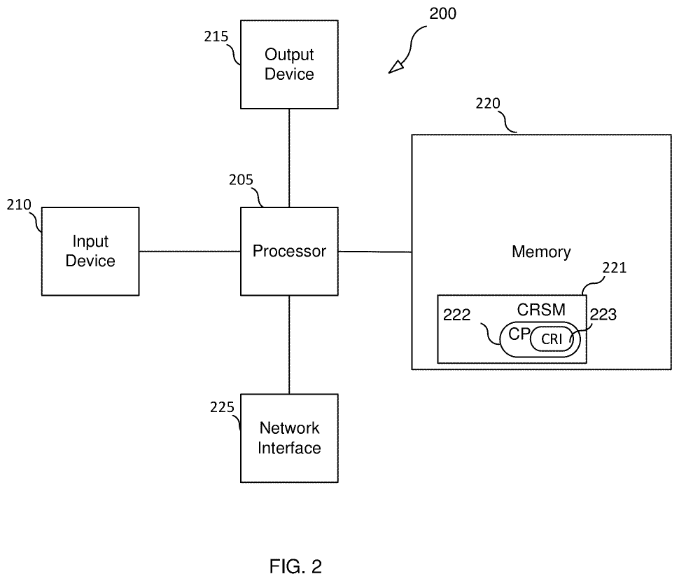

Referring to FIG. 2, a block diagram of a device 200, such as for example, user device 105, computer 120, tablet 125, and mobile device 130, in accordance with exemplary embodiments is illustrated. As shown in FIG. 2, the device 200 may include a processor 205, which may include one or more microprocessors and/or one or more circuits, such as an application specific integrated circuit (ASIC), field-programmable gate arrays (FPGAs), etc.

The device 200 may include a network interface 225. The network interface 225 is configured to enable communication with a communication network, using a wired and/or wireless connection. The network interface 225 may also be configured to enable communication with a wearable device 135, or the device 200 may include additional hardware for communicating with wearable device 135. The network interface 225 or additional hardware provided may enable, for example, Bluetooth communication for transmitting and receiving data wireless between the wearable device 135 and the user device 105.

The device 200 may include memory 220, such as non-transitive, which may include one or more non-volatile storage devices and/or one or more volatile storage devices (e.g., random access memory (RAM)). In instances where the device 200 includes a microprocessor, computer readable program code may be stored in a computer readable medium or storage memory 221, such as, but not limited to magnetic media (e.g., a hard disk), optical media (e.g., a DVD), memory devices (e.g., random access memory, flash memory), etc. The computer program 222 or software code can be stored on a tangible, or non-transitive, machine-readable medium or memory. In some embodiments, computer readable program code or instructions 223 is configured such that when executed by a processor, the code causes the device to perform the steps described below and herein. In other embodiments, the device is configured to perform steps described below without the need for code.

It will be recognized by one skilled in the art that these operations, algorithms, logic, method steps, routines, sub-routines, and modules may be implemented in software, in firmware, in special purpose digital logic, and any combination thereof without deviating from the spirit and scope of the embodiments disclosed herein.

The device 200 may include an input device 210. The input device is configured to receive an input from either a user or a hardware or software component. Examples of an input device 210 include a keyboard, mouse, microphone, touch screen and software enabling interaction with a touch screen, etc. The device can also include an output device 215. Examples of output devices 215 include monitors, televisions, mobile device screens, tablet screens, speakers, remote screens, etc. The output device 215 can be configured to display images, media files, text, or video, or play audio to a user through speaker output.

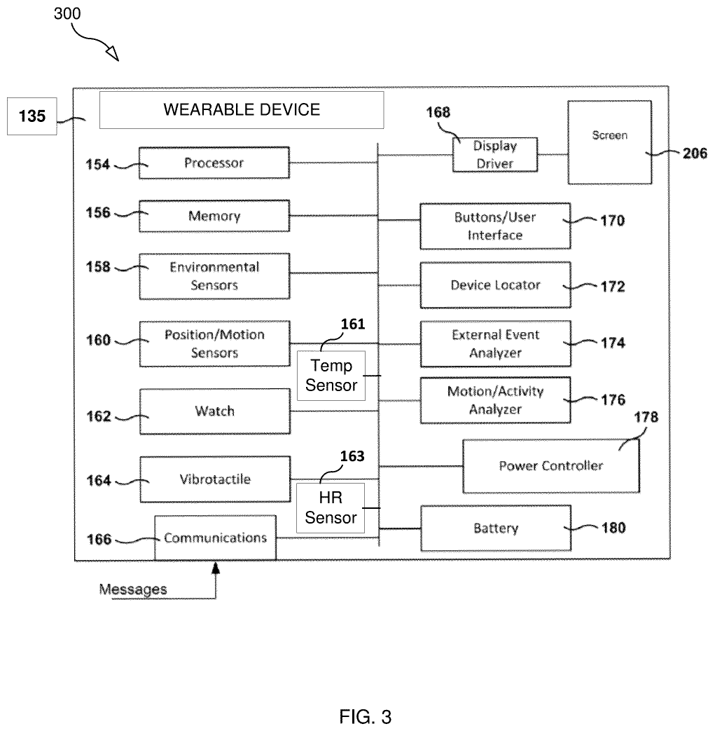

Referring now to FIG. 3, a block diagram of a wearable device 135 according to exemplary embodiments is illustrated. The wearable device 135 is an example of any of the wearable devices described herein, and including a watch, a fitness tracker with or without buttons, a ring, etc. The wearable device 135 includes processor 154, memory 156, one or more environmental sensors 158, one or more position and motion sensors 160, one or more temperature sensors 161, watch 162, one or more heart rate sensors 163, vibrotactile feedback module 164, display driver 168, touchscreen 206, user interface/buttons 170, device locator 172, external event analyzer 174, motion/activity analyzer 176, power controller 178, and battery 180, all of which may be coupled to all or some of the other elements within wearable device 135.

Examples of environmental sensors 158 include a barometric pressure sensor, a weather condition sensor, a light exposure sensor, a noise exposure sensor, a radiation exposure sensor, and a magnetic field sensor. Examples of a weather condition sensor include sensors for measuring temperature, humidity, pollen count, air quality, rain conditions, snow conditions, wind speed, or any combination thereof, etc. Examples of light exposure sensors include sensors for ambient light exposure, ultraviolet (UV) light exposure, or a combination thereof, etc. Examples of air quality sensors include sensors for measuring particulate counts for particles of different sizes, level of carbon dioxide in the air, level of carbon monoxide in the air, level of methane in the air, level of other volatile organic compounds in the air, or any combination thereof.

Examples of the position/motion sensor 160 include an accelerometer, a gyroscope, a rotary encoder, a calorie measurement sensor, a heat measurement sensor, a moisture measurement sensor, a displacement sensor, an ultrasonic sensor, a pedometer, an altimeter, a linear position sensor, an angular position sensor, a multi-axis position sensor, or any combination thereof, etc. In some embodiments, the position/motion sensor 160 measures a displacement (e.g., angular displacement, linear displacement, or a combination thereof, etc.) of the wearable device 135 over a period of time with reference to a three-dimensional coordinate system to determine an amount of activity performed by the user during a period of time. In some embodiments, a position sensor includes a biological sensor, which is further described below.

The vibrotactile module 164 provides sensory output to the user by vibrating portable device 152. Further, the communications module 166 is operable to establish wired or wireless connections with other electronic devices to exchange data (e.g., activity data, geo-location data, location data, a combination thereof, etc.). Examples of wireless communication devices include, but are not limited to, a Wi-Fi adapter, a Bluetooth device, an Ethernet adapter, and infrared adapter, an ultrasonic adapter, etc.

The touchscreen 206 may be any type of display with touch sensitive functions. In another embodiment, a display is included but the display does not have touch-sensing capabilities. The touchscreen may be able to detect a single touch, multiple simultaneous touches, gestures defined on the display, etc. The display driver 168 interfaces with the touchscreen 206 for performing input/output operations. In one embodiment, display driver 168 includes a buffer memory for storing the image displayed on touchscreen 206.

The buttons/user interface may include buttons, switches, cameras, USB ports, keyboards, or any other device that can provide input or output functions.

Device locator 172 provides capabilities for acquiring data related to the location (absolute or relative) of wearable device 135. Examples device locators 172 include a GPS transceiver, a mobile transceiver, a dead-reckoning module, a camera, etc. As used herein, a device locator may be referred to as a device or circuit or logic that can generate geo-location data. The geo-location data provides the absolute coordinates for the location of the wearable device 135. The coordinates may be used to place the wearable device 135 on a map, in a room, in a building, etc. in some embodiments, a GPS device provides the geo-location data. In other embodiments, the geo-location data can be obtained or calculated from data acquired from other devices (e.g., cell towers, Wi-Fi device signals, other radio signals, etc.), which can provide data points usable to locate or triangulate a location.

External event analyzer 174 receives data regarding the environment of the user and determines external events that might affect the power consumption of the user. For example, the external event analyzer 174 may determine low light conditions in a room, and assume that there is a high probability that the user is sleeping. In addition, the external event analyzer 174 may also receive external data, such as GPS location from a smart phone, and determine that the user is on a vehicle and in motion.

In some embodiments, the processor 154 receives one or more geo-locations measured by the device locator 172 over a period of time and determines a location of the wearable device 135 based on the geo-locations and/or based on one or more selections made by the user, or based on information available within a geo-location-location database of the network. For example, the processor 154 may compare the current location of the wearable device against known locations in a location database, to identify presence in well-known points of interest to the user or to the community. In one embodiment, upon receiving the geo-locations from the device locator 172, the processor 154 determines the location based on the correspondence between the geo-locations and the location in the geo-location-location database.

The one or more environmental sensors 158 may sense and determine one or more environmental parameters (e.g., barometric pressure, weather condition, amount of light exposure, noise levels, radiation levels, magnetic field levels, or a combination thereof, etc.) of an environment in which the wearable device is placed.

The one or more temperature sensors 161 may sense and determine the wearer's temperature, such as for example by having the one or more temperature sensors in contact with the wearer's skin.

The watch 162 is operable to determine the amount of time elapsed between two or more events. In one embodiment, the events are associated with one or more positions sensed by the position sensor 160, associated with one or more environmental parameters determined by the environmental sensor 158, associated with one or more geo-locations determined by the device locator 172, and/or associated with one or more locations determined by the processor 154.

The one or more heart rate sensors 163 may sense the heartbeat of the person wearing the wearable device 135 and determine the wearer's heart rate. This information can be used for various purposes, such as monitoring exercise intensity, tracking sleep patterns, and detecting signs of stress. Power controller 178 manages and adjusts one or more power operational parameters defined for the wearable device 135. In one embodiment, the power operational parameters include options for managing the touchscreen 206, such as by determining when to turn ON or OFF the touchscreen, scan rate, brightness, etc. In addition, the power controller 178 is operable to determine other power operational parameters, besides the parameters associated with the touchscreen, such as determining when to turn ON or OFF other modules (e.g., GPS, environmental sensors, etc.) or limiting the frequency of use for one or more of the modules within wearable device 135.

Wearable device 135 may have a variety of internal states and/or events which may dynamically change the characteristics of the touchscreen or of other modules. These states may include one or more of the following:
Battery level
Notifications/Prompting of user interaction
   Alarm
   Timer elapsed
   Email received/sent
   Instant Message received/sent
   Text message received/sent
   Calendar event
   Physiological goal met (e.g., 10,000 steps reached in the day)
   Non-physiological goal met (e.g., completed a to-do item)
   Application notifications
   Music player notifications (e.g., song ended/started, playlist ended/started)
User Interface
   Layout of virtual buttons on the touchscreen
   Expected user interaction based on what is displayed and/or the application in the foreground of the operating system.
      Expected user touch speed (e.g., fast for typing or playing a game, slow for reading an article)
      Expected user touch area
      Expected user touch trajectory (e.g., some games require long, straight swipes, while applications that take text input may require a touch to one specific area with little or no trajectory).
User interaction through non-touchscreen inputs
   User pressing a button
   User touching a capacitive touch sensor not integrated into the touchscreen
   User activating a proximity sensor
   Sensors which detect the user attempting to interact with the screen
      Force transducer under the screen
      Gyroscope, magnetometer, and/or accelerometer located near the screen
      Pressure transducer to measure change in pressure due to housing deflection when user presses on or near the screen
      Tap or initial touch detection using one or more or a combination of: accelerometers, piezoelectric sensors, motion sensors, pressure sensors, force sensors
User Characteristics
   Gender
   Age
   Weight
   Geographical location (e.g., urban area, rural area, country, city, town, exact address and or GPS coordinates)
      Current location
      Residence location
      Work location

It is noted that these states may be communicated to the user through one or more methods including, but not limited to, displaying them visually, outputting an audio alert, and/or haptic feedback.

In some embodiments, data analysis of data produced by different modules may be performed in wearable device 135, in other device in communication with wearable device 135, or in combination of both devices. For example, the wearable device may be generating a large amount of data related to the heart rate of the user. Before transmitting the data, the wearable device 135 may process the large amount of data to synthesize information regarding the heart rate, and then the wearable device 135 may send the data to a server that provides an interface to the user. For example, the wearable device may provide summaries of the heart rate in periods of one minute, 30 seconds, five minutes, 50 minutes, or any other time period. By performing some calculations in the wearable device 135, the processing time required to be performed on the server is decreased.

Some other data may be sent in its entirety to another device, such as steps the user is taken, or periodical updates on the location of the wearable device 135. Other calculations may be performed in the server, such as analyzing data from different modules to determine stress levels, possible sickness by the user, etc.

It is noted that the embodiments illustrated in FIG. 3 are exemplary. Other embodiments may utilize different modules, additional modules, or a subset of modules. In addition, some of the functionality of two different modules might be combined in a single module, or the functionality of a single module might be spread over a plurality of components. The embodiments illustrated in FIG. 3 should therefore not be interpreted to be exclusive or limiting, but rather exemplary or illustrative.

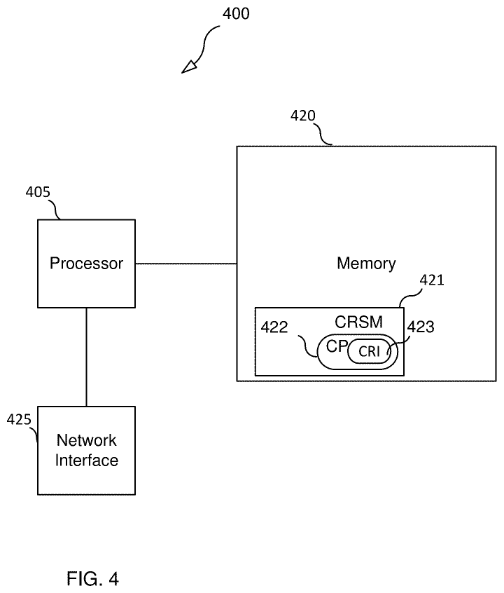

FIG. 4 is a block diagram of a remote device in accordance with exemplary embodiments is illustrated. As shown in FIG. 4, the remote device 400 may include a network interface 425 for transmitting and receiving data, a processor 405 for controlling operation of the server device 400, and a memory 420 for storing computer readable instructions (e.g., software) and data. The network interface 425 and memory 420 are coupled to and communicate with the processor 405. Processor 405 controls the operation of network interface 425 and memory 420 and the flow of data and functionality between them. In various embodiments inputs can come from the device 200, to the remote device 400, via the network interface 425. Processing can occur at the remote device 400, at the device 200, or at both. In various embodiments, remote device 400 may be a server.

Processor 405 may include one or more microprocessors, and/or one or more circuits, such as an application specific integrated circuit (ASIC), field-programmable gate arrays (FPGAs), etc. Network interface 425 can be configured to enable communication with a communication network, using a wired and/or wireless connection. Memory 420 can include one or more non-volatile storage devices and/or one or more volatile storage devices (e.g., random access memory (RAM)). In instances where remote device 400 includes a microprocessor, computer readable program code may be stored in a computer readable medium or storage memory 421, such as, but not limited to magnetic media (e.g., a hard disk), optical media (e.g., a DVD), memory devices (e.g., random access memory, flash memory), etc. The computer program 422 or software code can be stored on a tangible, or non-transitive, machine-readable medium or memory. In some embodiments, computer readable program code or instructions 423 is configured such that when executed by a processor, the code causes the device to perform the steps described below and herein. In other embodiments, the device is configured to perform steps described below without the need for code.

It will be recognized by one skilled in the art that these operations, logic, method steps, routines, algorithms, sub-routines, and modules may be implemented in software, in firmware, in special purpose digital logic, and any combination thereof without deviating from the spirit and scope of the embodiments disclosed herein.

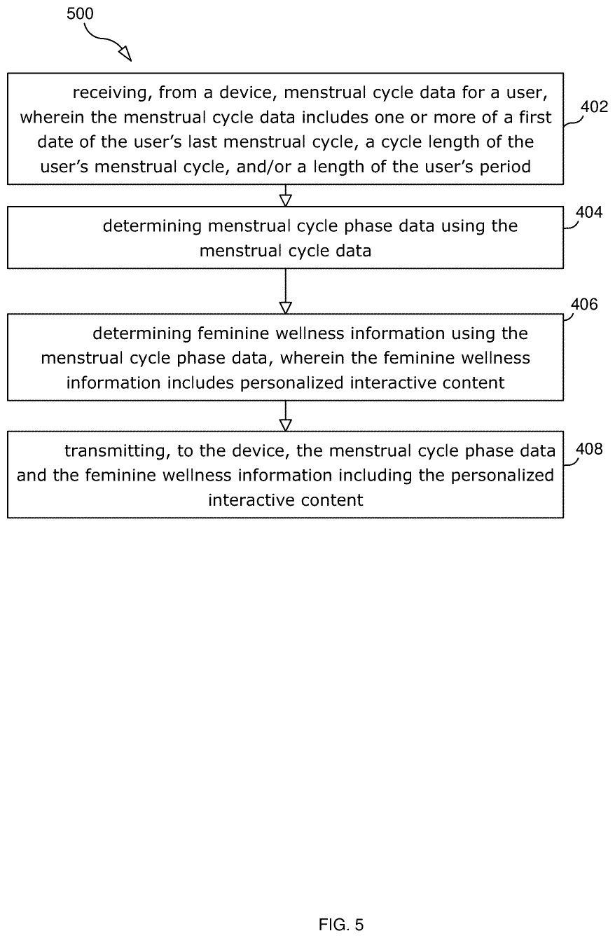

FIG. 5 is a flow diagram illustrating steps of an exemplary method 400 for providing feminine wellness information to one or more users.

In some embodiments, method 500 includes, receiving, from a device, menstrual cycle data from a user (step 502). The menstrual cycle data may include a first date of a last menstrual cycle or a cycle start date. It may also include an average cycle length or a cycle duration, an average length of menstrual phase, an average period length or period duration, the user's time zone, and a current date.

The method further includes determining menstrual cycle phase data using the menstrual cycle data (step 504). The menstrual cycle phase data may include whether the user is in one of the following phases: Menstrual, Follicular, Ovulation, Luteal. The menstrual cycle phase data may also include the day that the user is in within their cycle, the day that the user is in within the phase, and whether the user is in a beginning, a middle, or an end of one of the phases. The menstrual cycle phase data may further include information regarding different changes to the mood and body during each phase.

The method further includes determining feminine wellness information using menstrual cycle phase data (step 506). The feminine wellness information may include customized streaming workouts, nutrition profiles, recipes, and science-based “horoscope-like” insights into a user's emotions that are unique to each phase and may be unique to each day.

The method further includes transmitting, to the device, the menstrual cycle phase data and the feminine wellness information (step 508).

In another aspect of the disclosed embodiments, a method is implemented in a user device. The user is prompted to input menstrual cycle data. The menstrual cycle data may include a first date of a last menstrual cycle. It may also include average cycle length, average length of menstrual phase, an average period duration, and the user's time zone. The user device is configured to transmit the inputted data to, for example, a communication network. The user data is processed in accordance with the above-described method of FIG. 5. The user device is further configured to receive menstrual cycle phase data and the feminine wellness information, and to display, on the device, the menstrual cycle phase data and the feminine wellness information. The feminine wellness information may include customized streaming workouts, nutrition profiles, recipes, and science-based “horoscope-like” insights into a user's emotions that are unique to each phase and may be unique to each day.

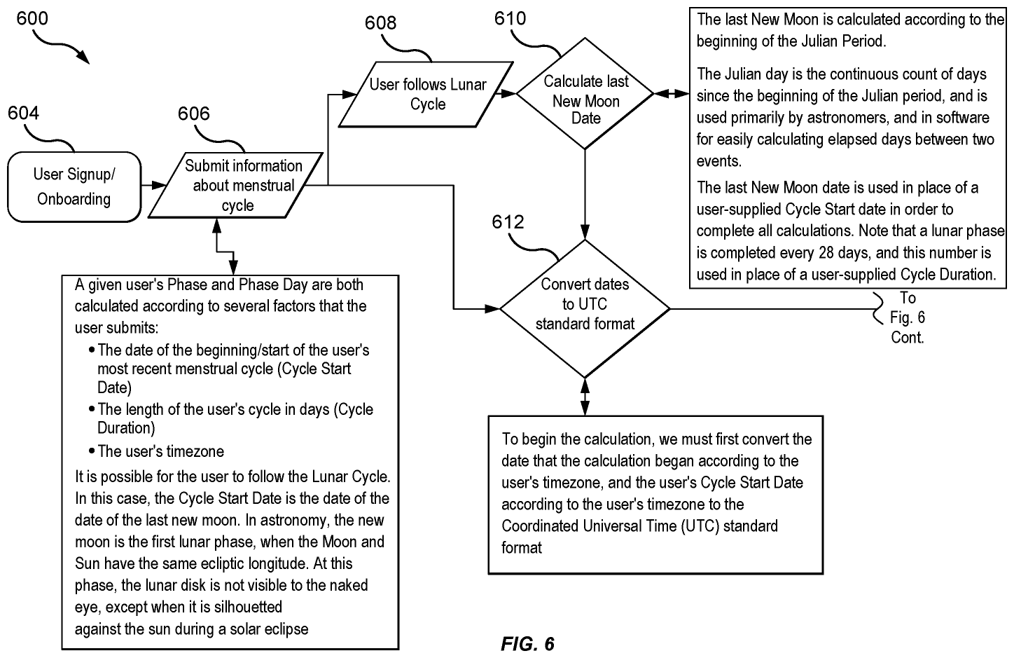

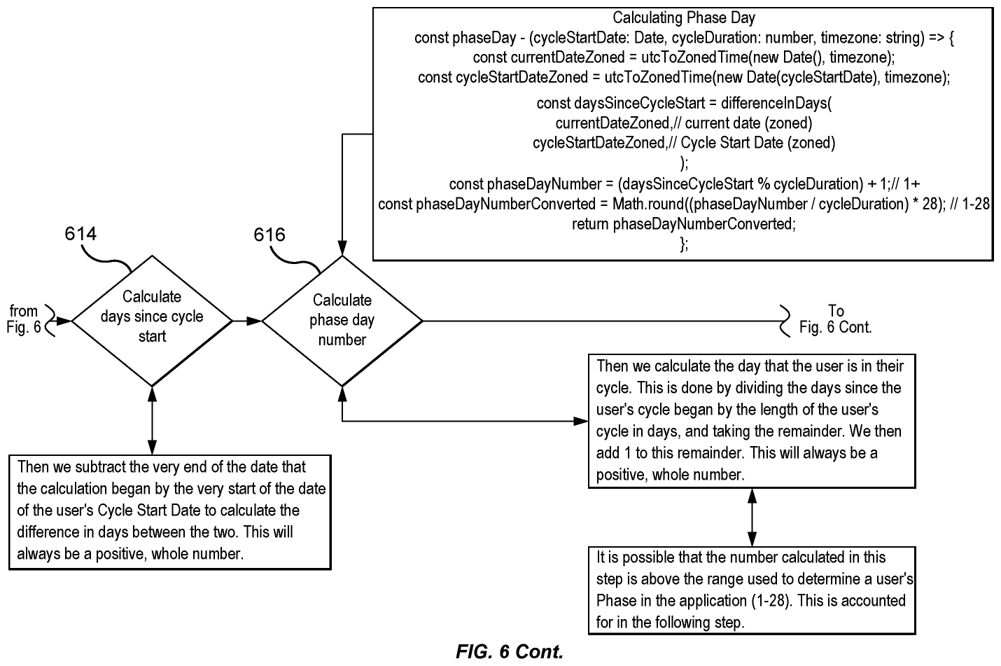

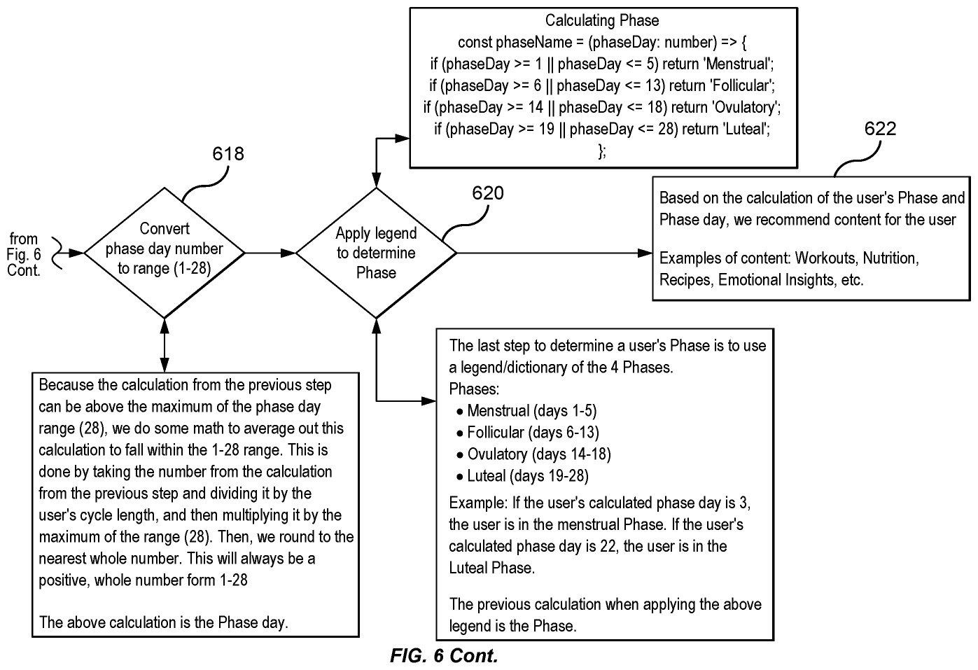

FIG. 6 is a block diagram of an exemplary architecture according to exemplary embodiments for providing feminine wellness information to one or more users.

In some embodiments, architecture 600 includes onboarding a user (604). Onboarding a user may include displaying on a device a request for the user's name and email address and receiving from the user the user's name and email address. Onboarding may further include authenticating the user's email address by transmitting a verification code to the user's email address and receiving the verification code as an input from the user. Onboarding may further include receiving from the user the user's time zone. In some embodiments, the user may select the time zone from a dropdown menu.

The architecture further includes receiving, from a device, menstrual cycle data from a user (606). Menstrual cycle data may include the date of the beginning or start of the user's most recent menstrual cycle or cycle start date and the length of the user's cycle in days or cycle duration. The method may include displaying on the device a calendar or other selection of dates. The user may select and submit from the calendar a first date of a last menstrual cycle. The method may also include displaying on the device a slide bar to select an average period length and an average cycle duration. The method may include receiving a first set of menstrual cycle data. The method may further include receiving an updated set of menstrual cycle data.

The architecture may include displaying on the device an option to follow a lunar cycle (608). The user may follow the lunar cycle if she experiences irregular or no period. In this embodiment, the cycle start date is a date of a last new moon. The architecture may include calculating the date of the last new moon (610). A new moon phase is a first lunar phase when the moon and sun have the same ecliptic longitude. During the new moon phase, a lunar disk is not visible to a naked eye, except when a lunar disk is silhouetted against a sun during a solar eclipse. The date of the last new moon is calculated according to a beginning of a Julian period. A Julian day is a continuous count of days since the beginning of the Julian period. The date of the last new moon is used in place of a user-supplied cycle start date in order to complete all calculations. A lunar phase is completed every 28 days, and this number is used in place of a user-supplied cycle duration.

The architecture may further include converting the cycle start date (i.e., the user-supplied cycle start date or the date of the last new moon) and a calculation start date (e.g., a current date or date that the calculation begins) according to the user's time zone to a cycle start date and a calculation start date according to the Coordinated Universal Time (UTC) standard format (612).

The architecture may further include calculating a number of days since the cycle start date (614). The number of days since the cycle start date is the difference between the calculation start date and the cycle start date. The method may include subtracting the calculation start date from the cycle start date, which will always be a positive whole number.

The architecture may further include calculating a phase day number (616). The phase day number is a day that the user is in their cycle. The method may include dividing the number of days since the cycle start date by the cycle duration (e.g., the user-supplied cycle duration or 28-days of the lunar phase). The method further includes adding 1 to the remainder, which will always result in a positive, whole number.

The architecture may further include converting the phase day number to a phase day (618). The phase day is a day or number within a phase day range (e.g., 1-28). The method may include dividing the phase day number by the user's cycle duration, multiplying by a maximum of the phase day range (e.g., 28), and then rounding to a nearest whole number. The phase day will always be a positive, whole number within the phase day range.

In some embodiments, the phase day may be calculated as follows:
Phase Day—Converting phase day number based on the user's cycle Start Date, Cycle Duration, and Time zone. If the user has a cycle duration that's not 28, the method to calculate her phase day is round (phasedaynumber/cycleduration)*28, which gives us the phase relative to when the user was in a 28 day cycle curation (e.g. If a user has 32 days, and she's on day 18, then phase day: round(18/32*28)=16, which means her day 18 equals to day 16 if she was on a 28 day cycle

const phaseDay = (cycleStartDate: Date, cycleDuration: number, timezone:
string) => {
const currentDateZoned = utcToZonedTime(new Date( ),timezone);
const cycleStartDateZoned = utcToZonedTime(new
Date(cycleStartDate),timezone);
const daysSinceCycleStart = differenceInDays (
currentDateZoned, //current date (zoned)
cycleStartDateZoned, // Cycle Start Date (zoned)
);
const phaseDayNumber = (daysSinceCycleStart % cycleDuration) + 1; // 1+
const phaseDayNumberConverted = Math.round((phaseDayNumber /
cycleDuration * 28)); // 1-28
return phaseDayNumberConverted;
};

The architecture may further include determining a user's phase (step 620). The method may also include determining a user's day within the phase. A user's phase may be determined using a legend or dictionary of the four (4) phases:
Menstrual or “Restore” phase (days 1-5)
Follicular or “Awaken” phase (days 6-13)
Ovulatory or “Perform” phase (days 14-18)
Luteal or “Balance” phase (days 19-28)

For example, if the user's phase day is 3, the user is in the Menstrual Phase. If the user's phase day is 22, the user is in the Luteal Phase.

In some embodiments, the phase may be calculated as follows:
If the user's phase day is 1-5 then she is in the Menstrual phase
If the user's phase day is 6-13 then she is in the Follicular phase
If the user's phase day is 14-18 then she is in the Ovulatory phase
If the user's phase day is 19-28 then she is in the Luteal phase
Example: If the user's calculated phase day is 3, the user is in the Menstrual Phase. If the user's calculated phase day is 22, the user is in the Luteal Phase.

const phaseName = (phaseDay: number) => {
if (phaseDay >= 1 || phaseDay <= 5) return ‘Menstrual’;
if (phaseDay >= 6 || phaseDay <= 13) return ‘Follicular’;
if (phaseDay >= 14 || phaseDay <= 18) return ‘Ovulatory’;
if (phaseDay >= 19 || phaseDay <= 28) return ‘Luteal’;
};

The architecture may further include determining recommended content for the user based on the user's phase (622). Content may include workouts, nutrition, recipes, emotional insights, or other feminine wellness information.

In some embodiments, the phase day and phase are determined by mathematical calculation using (day in cycle/cycle duration)*28 (rounded). If a user has exactly 28 days cycle length, and the user is in day 7, the “Snatched Abs” video will be curated and presented to the user in the “Today's workout” in the Workout tab. If a user has less than 28 day cycle length, say 25 days, and she's on day 5 the system will calculate which day would it be if the user is on a normal 28 day cycle to determine what video should be played. Specifically, 5/25*28=5.6 and we will round it up to day 6, and hence day 6 video will be played.

If a user has more than 28 day cycle length, say 35 days, and she's on day 3, the calculation will be 3/35*28=2.4 and we will round it down to 2 and system will play day 2 video in the sheet above.

Also, “additional phase days” column indicates what other phase days can we play this video specifically when user has longer than 28 days cycle length. E.g. “30 min Restorative Detox” can also be played for day 2 and 3 if the user has longer phase days than 5.

The same video can be recycled, but the system will NOT play the same video on 2 consecutive days, as discussed below.

In some embodiments, the recommended content is determined in view of the freshness. In some embodiments, the same video may not be played on two consecutive days. For example, the following computer program code can be used to ensure that the recommended content served to the user through, for example, a graphical user interface (GUI), has not been played for the user on 2 consecutive days:

```
// get random workout
const [randomWorkout] = await prisma.$transaction([
  prisma.workout.findFirstOrThrow({
    where: {
      id: { not: currentDashboard.workout.id }, // dont show the
same workout 2 days in a row
      phasedays: { some: { phaseday: { number:
phasedayNumber } } },
      published: true, // ensure that the workout is published
    },
    skip: randomNumber(workoutCount),
    take: 1,
  }),
]);
```

This same logic can be applied for other content, insights and nutrition food recommendations as well.

The following table provides further details of the architecture 600 illustrated in FIG. 6:

| | |
|---|---|
| 606 - Submit information | A given user's Phase and Phase Day are both calculated |
| about menstrual cycle | according to several factors that the user submits |
| | The date of the beginning/start of the user's most |
| | recent menstrual cycle (Cycle Start Date) |
| | The length of the user's cycle in days (Cycle |
| | Duration) |
| | The user's timezone |
| | It is possible for the user to follow the Lunar Cycle. In this |
| | case, the Cycle Start Date is the date of the last new |
| | moon. In astronomy, the new moon is the first lunar |
| | phase, when the Moon and Sun have the same ecliptic |
| | longitude. At this phase, the lunar disk is not visible to the |
| | naked eye, except when it is silhouetted against the Sun |
| | during a solar eclipse. |
| 610 - Calculate last New | The last New Moon is calculated according to the |
| Moon date | beginning of the Julian Period. The Julian day is the |
| | continuous count of days since the beginning of the |
| | Julian period, and is used primarily by astronomers, and |
| | in software for easily calculating elapsed days between |
| | two events. The last New Moon date is used in place of a |
| | user-supplied Cycle Start date in order to complete all |
| | calculations. Note that a lunar phase is completed every |
| | 28 days, and this number is used in place of a user- |
| | supplied Cycle Duration. |
| 612 - Convert dates to | To begin the calculation, we must first convert the date |
| UTC standard format | that the calculation began according to the user's |
| | timezone, and the user's Cycle Start Date according to |
| | the user's timezone to the Coordinated Universal Time |
| | (UTC) standard format. |
| 614 - Calculate days | Then we subtract the very end of the date that the |
| since cycle start | calculation began by the very start of the date of the |
| | user's Cycle Start Date to calculate the difference in days |
| | between the two. This will always be a positive, whole |
| | number |
| 616 - Calculate phase day | Then we calculate the day that the user is in their cycle. |
| number | This is done by dividing the days since the user's cycle |
| | began by the length of the user's cycle in days, and |
| | taking the remainder. We then add 1 to this remainder. |
| | This will always be a positive, whole number. |
| | It is possible that the number calculated in this step is |
| | above the range used to determine a user's Phase in the |
| | application (1-28). This is accounted for in the following |
| | step. |
| 618 - Convert phase day | Because the calculation from the previous step can be |
| number to range (1-28) | above the maximum of the phase day range (28), we do |
| | some math to average out this calculation to fall within |
| | the 1-28 range. This is done by taking the number from |
| | the calculation from the previous step and dividing it by |
| | the user's cycle length, and then multiplying it by the |
| | maximum of the range (28). Then, we round to the |
| | nearest whole number. This will always be a positive, |
| | whole number from 1-28. The above calculation is the |
| | Phase Day |
| 620 - Apply legend to | The last step to determine a user's Phase is to use a |
| determine Phase | legend/dictionary of the 4 Phases. |
| | Phases |
| | Menstrual (days 1-5) |
| | Follicular (days 6-13) |
| | Ovulatory (days 14-18) |
| | Luteal (days 19-28) |
| | Example If the user's calculated phase day is 3, the user |
| | is in the Menstrual Phase. If the user's calculated phase |
| | day is 22, the user is in the Luteal Phase. The previous |
| | calculation when applying the above legend is the Phase. |
| 622 | Based on the calculation of the user's Phase and Phase |
| | day, we recommend content for the user. Examples of |
| | content Workouts, Nutrition, Recipes, Emotional Insights, |
| | etc. |

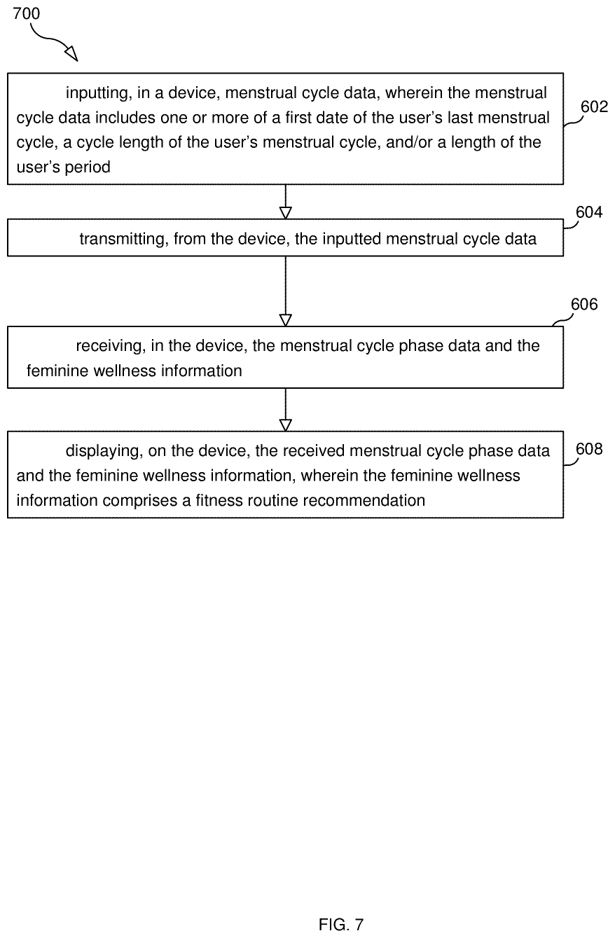

FIG. 7 is a flow diagram illustrating steps of an exemplary method 700 for providing feminine wellness information to one or more users.

In some embodiments, method 700 includes inputting, in a device, menstrual cycle data, wherein the menstrual cycle data includes a first date of a last menstrual cycle (step 702). The method further includes transmitting, from the device, the inputted menstrual cycle data (step 704). The method further includes receiving, in the device, the menstrual cycle phase data and the feminine wellness information (step 706). The method further includes displaying, on the device, the received menstrual cycle phase data and the feminine wellness information, wherein the feminine wellness information comprises a fitness routine recommendation (step 708).

In some embodiments, a cycle syncing content recommendation engine is used. The process for curating the content (e.g., video, text, etc.) based on the cycle and other info on the user can be recommended based on the following computer code. In one embodiment, the computer code used is the following:

```
export const userPhasedayNumber = (
  cycleStart: Date,
  cycleDuration: number,
  timeZone: string,
  menstrualPhaseLength?: number,
) => {
  if (!menstrualPhaseLength) {
    menstrualPhaseLength = DEFAULT_MENSTRUAL_PHASE_LENGTH;
  }
  const rightNowZoned = utcToZonedTime(new Date( ), timeZone);
  const cycleStartZoned = utcToZonedTime(new Date(cycleStart), timeZone);
  const daysSincePeriodStart = differenceInDays(endOfDay(rightNowZoned),
startOfDay(cycleStartZoned));
  const phasedayNumberRaw = getRawPhaseday(daysSincePeriodStart,
cycleDuration);
  const phasedayNumber = phaseDayNumberFromRaw(phasedayNumberRaw,
cycleDuration, menstrualPhaseLength);
  return phasedayNumber;
};
```

In another embodiment, the computer code used is the following:

```
export const phaseDayNumberFromRaw = (
  phaseDayNumberRaw: number,
  cycleDuration: number,
  menstrualPhaseLength: number,
) => {
  // if phaseDayNumber raw is in the user's menstrual cycle, return the number
between 1 and 5 that's closest
  if (menstrualPhaseLength >= phaseDayNumberRaw) {
    const menstrualPhaseMultiplier = DEFAULT_MENSTRUAL_PHASE_LENGTH
/ menstrualPhaseLength;
    const menstrualPhaseDayRaw = menstrualPhaseMultiplier *
phaseDayNumberRaw;
    //? Math.min here to make sure we're never over 5 so that we're always
showing ‘Menstrual’ content when relevant
    const menstrualPhaseDay = Math.min(Math.round(menstrualPhaseDayRaw),
DEFAULT_MENSTRUAL_PHASE_LENGTH);
    return menstrualPhaseDay;
  }
  const DEFAULT_REST_OF_CYCLE_LENGTH =
DEFAULT_CYCLE_DURATION − DEFAULT_MENSTRUAL_PHASE_LENGTH;
  const userRestOfCycleLength = cycleDuration − menstrualPhaseLength;
  //? So if the user isn't in the menstrual phase, we have to figure out where she is
  //? in the restOfCycle phase and map that to our 28 day value
  const restOfPhaseMultiplier = DEFAULT_REST_OF_CYCLE_LENGTH /
userRestOfCycleLength;
  const restOfPhasedayRaw = phaseDayNumberRaw − menstrualPhaseLength;
  const restOfPhaseday = Math.round(restOfPhaseMultiplier *
restOfPhasedayRaw);
  const phasedayNumber = restOfPhaseday +
DEFAULT_MENSTRUAL_PHASE_LENGTH;
  return phasedayNumber;
};
export const userPhasedayNumberRaw = (cycleStart: Date, cycleDuration:
number, timeZone: string) => {
  const rightNowZoned = utcToZonedTime(new Date( ), timeZone);
  const cycleStartZoned = utcToZonedTime(new Date(cycleStart), timeZone);
  const daysSincePeriodStart = differenceInDays(endOfDay(rightNowZoned),
startOfDay(cycleStartZoned));
  return getRawPhaseday(daysSincePeriodStart, cycleDuration);
};
```

These algorithms, by taking in any specific start date of a user's menstrual cycle, a user's specific menstrual cycle length and the length of a user's period, can assign that user a number between 1 and 28. This number is then used to find curated content that is specifically targeted to be as relevant as possible for where a user is in her menstrual cycle.

In some embodiments, these algorithms may be used to calculate an average menstrual cycle duration and an average period duration for a user. The average menstrual cycle duration and average period duration for a user may be used, in conjunction with the first day of the last menstrual cycle or phase for the user, to determine the menstrual cycle phase data and the feminine wellness information to be transmitted to the user's device.

The calculated number may be used to then find content. In some embodiments, there is a content library of all the workout videos, nutrition, recipes, insights, and content is uploaded on a regular basis. Once the number of the phase day is defined, the system will determine what phase day is supposed to be on a 28 days cycle if the user does not have 28 days as cycle duration.

In some embodiments, each video is tagged with the number of days that it could be used and that video can be pulled if the user has a phase day that equals that number, and the system will prepare that video and display it on the Workout screen. Same process applies to other content as well.

In some embodiments, the tagging based on the day allocation is arranged as follows:

| | | | Additional | Video Asset |
|---|---|---|---|---|
| Video Title | Phase | Day | Phase Days | Link |
| 30 min Restorative Detox | Restore | 1 | | 44595 |
| 20 min Stretch to De-Stress | Restore | 2 | 1, 2, 3, | |
| Lymph Flow Detox | Restore | 3 | 1, 2, | |
| Slim Thighs and Booty | Restore | 4 | | 5 |
| Restorative Mat Pilates | Restore | 5 | | 44624 |
| Hips Like Venus | Awaken | 6 | | 7 |
| Snatched Abs | Awaken | 7 | | 6 |
| Core Full Body | Awaken | 8 | | 7 |
| Upper Body Sculpt | Awaken | 9 | | 44750 |
| Full Body Transformation | Awaken | 10 | 7, 8, | |
| Inner and Outer | Awaken | 11 | | 10 |
| Thigh Stability | | | | |
| Strong & Graceful | Awaken | 12 | | 44750 |
| Core and Abs | | | | |
| Inner Thigh Lengthening | Awaken | 13 | | 44753 |
| Movement to Awaken | Perform | 14 | | 17 |
| Your Sensuality | | | | |
| Brazilian Booty HIIT | Perform | 15 | 16, 14 | |
| Aerobic Fat Burn Cardio | Perform | 16 | 17, 18 | |
| Kickboxing Cardio | Perform | 17 | 16, 18 | |
| Cardio Boxing with Circuits | Perform | 18 | 16, 17 | |
| Cardio Barre and Sculpt | Balance | 19 | 21, 22, 20 | |
| Upper Body Sweat and Abs | Balance | 20 | 19, 22 | |
| Lengthening and | Balance | 21 | 27, | |
| Strengthening | | | | |
| Balance and Stability | Balance | 22 | 21, 25 | |
| | | | 19, 20, 21, | |
| Impossible Butt Lift | Balance | 23 | 22 | |
| Yin Yoga for PMS | Balance | 24 | | 28 |
| Balance and Posture | Balance | 25 | 22, 24 | |
| Corrective | | | | |
| Body Positive Pilates | Balance | 26 | | 28 |
| Inner/Outer Thigh Tone | Balance | 27 | | 21 |
| PMS Pilates | Balance | 28 | 24, 26, 27 | |

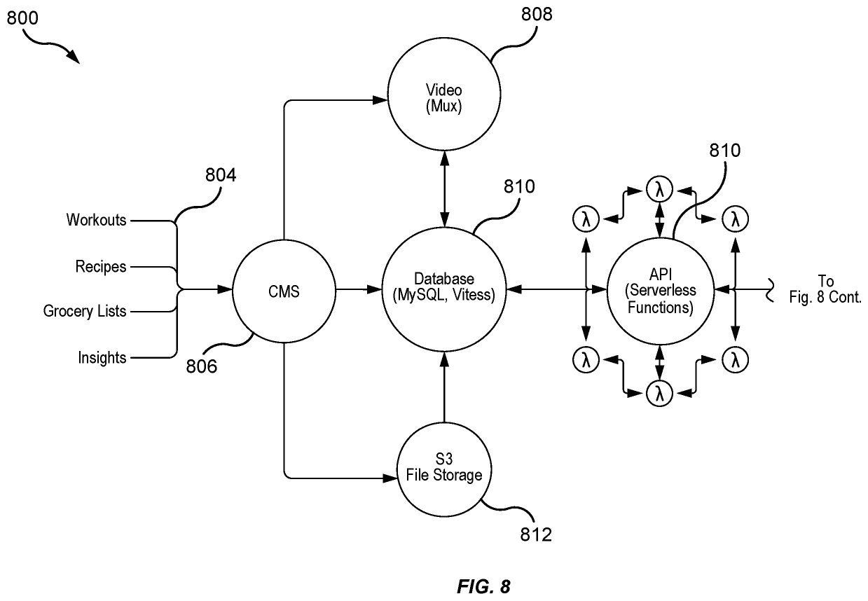

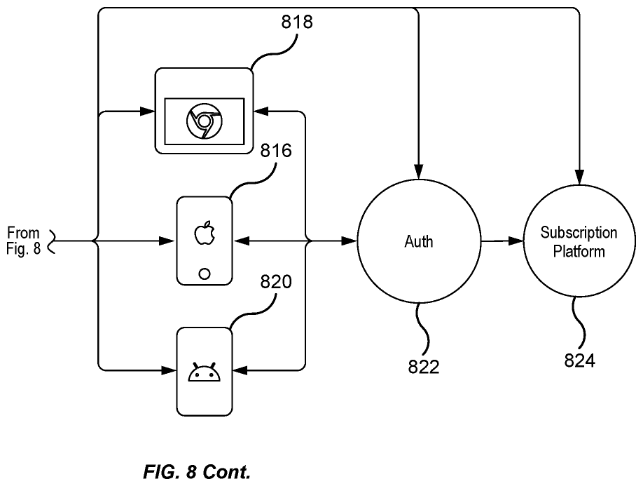

FIG. 8 is a block diagram of an exemplary architecture 800 for client-service interaction illustrating the server/cloud side systems and the user/client facing devices.

In some embodiments, data/content/information 804 relating to feminine wellness information, including, for example, workouts, recipes, grocery lists and insights, is input or transmitted to a content management system (CMS) 706.

CMS 806 is configured to communicate with a video component/module 808, such as a video multiplexer (mux) 808, a database component/module 810, such as MySQL, Vitess or Planetscale, and a file storage component/module 812, such as Amazon S3. The CMS 806 is configured to provide data/content/information to the video component/module 808, the database component/module 810, and the file storage component/module 812.

The database module/component 810 receives information from the file storage component/module 812. The video component/module 808 receives information from and sends information to the database module/component 810.

The database component/module 810 is configured to communicate with one or more application programming interfaces (APIs) 814 which may provide serverless functions, such as, for example, Amazon Web Service (AWS) Lambda. The APIs 814 are configured to communicate with user devices or nodes, such as, for example, an Apple iPhone smartphone 816 or like device, an Apple iPad tablet 818 or like device, or an Android smartphone 820 or like device.

As part of the process of preparing and delivering the customized feminine wellness information to a user's device (816, 818 or 820) or node, the user's subscription to the service can be authenticated by the Authentication (Auth) component/module 822. User information, such as, for example, a username and a password, can be input through a graphical user interface (GUI) enabled by an application or through a web browser on the user's device (816, 818, or 820) or node, and the user information can be transmitted to the Auth component/module 822. The authentication performed by the Auth component/module 822 may include transmitting a verification code to the user's email address and receiving the verification code as an input from the user.

The Auth component/module 822 is configured to communicate with a Subscription Platform component/module 824. If the Auth component/module 822 authenticates the user, based on the user's username, such as the user's email address, and a password, for example, the Auth component/module 822 will transmit the authentication information to the Subscription Platform component/module, which will then authorize the provision of the customized feminine wellness information that was generated for the specific user based on the menstrual cycle data for that user. Menstrual cycle data may include a date of a beginning or start of the user's most recent menstrual cycle or a cycle start date and a length of the user's cycle in days or a cycle duration. Menstrual cycle data may include the length of a user's period. Menstrual cycle data may also include average menstrual cycle duration and an average period duration for a user. The average menstrual cycle duration and average period duration for a user may be used, in conjunction with the first day of the last menstrual cycle or phase for the user, to determine the menstrual cycle phase data and the feminine wellness information. The feminine wellness information may include workouts, recipes, grocery lists, and insights.

The customized feminine wellness information generated for the specific user is based on the user's menstrual cycle phase. For example, if the user is in Restore (Menstrual) phase, levels of estrogen and progesterone are typically at their lowest, and women could experience low energy and cramps as the body sheds the uterus lining. Hence, the method recommends gentle workouts, and the system will recommend the best workout for that phase day.

If the user is in Awaken (Follicular) phase, hormone levels are rising, estrogen rises during this phase, and typically women will experience more energy and the method recommends workouts that are moderate intensity.

If the user is in Perform (Ovulatory) phase, estrogen and level is at its peak, energy and sex drive are at all-time high and the method recommends workouts that are higher intensity.

If the user is in Balance (Luteal) phase, estrogen level can rise again and progesterone at its peak during the first half of the phase, and progesterone level will drop on the second half of the phase due to menstruation approaching. The workouts recommended for a first half of the phase are different from a second half of the phase. The method recommends moderate workouts for the first half and more gentle workouts for the second half as women start experiencing PMS symptoms.

The same feminine wellness information can be transmitted for multiple days within a cycle.

In an exemplary embodiment, a software application is provided that is implemented in the user device 105 and that is configured to communicate via a network 104 with a remote device 110. The remote device 110 is configured with a software application that processes the data and other information received from the user device 105 and provides to the user device 105 feminine wellness information, as described above. An exemplary architecture for these software applications is illustrated in FIG. 6 and described above. An exemplary architecture for client-service interaction in connection with implementation of these software applications is illustrated in FIG. 8 and described above.

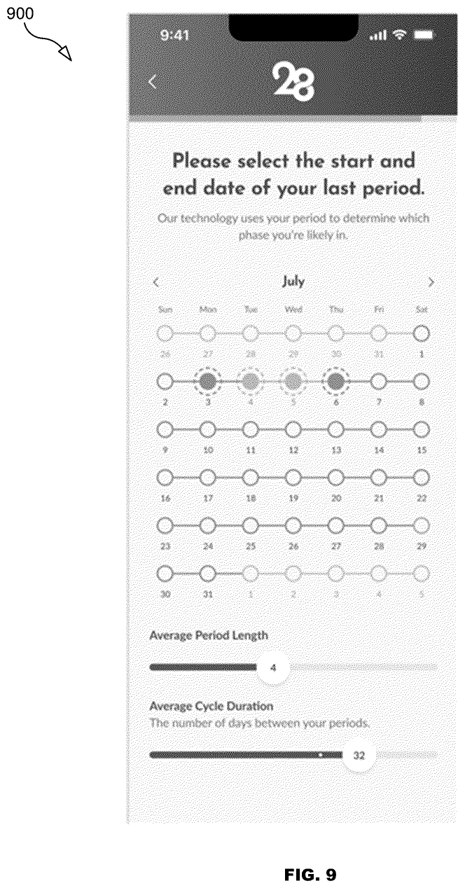

In an exemplary embodiment, the user is prompted to input menstrual cycle data. The menstrual cycle data may include a first date of a last menstrual cycle. Menstrual cycle data may include the length of a user's period. It may also include average cycle length, average length of menstrual phase, and the user's time zone. FIGS. 9-11 illustrate exemplary screenshots from an application displayed on a user device showing this functionality.

Referring to FIG. 9, screenshot 900 shows a user submitting menstrual cycle information. In this step, user can:
1. Choose the first day of last period
2. Slide the bar left and right to adjust the average cycle duration that's OTHER than 28 days (e.g., if user has 24 cycle days, she can slide left to 24 and save the information)
Why: User can update these 2 pieces of information whenever after they signup in the “Profile tab as below
How: The purpose of this is to predict which day throughout cycle the user is in and which phase she is in. Especially because users can choose cycle duration days other than 28 as not everyone has 28 days cycle
For example, if user chooses December 9 as the first day of the last period and an average cycle duration of 26 days, then system will predict that the 1st day of the next menstrual phase will be on January 4.

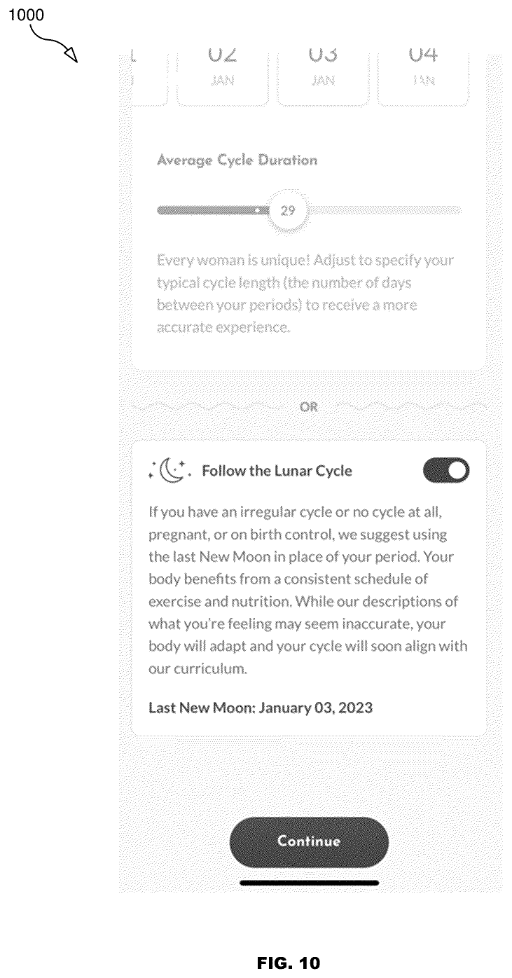

Referring to FIG. 10, screenshot 1000 shows a user choosing Lunar Cycle. submitting menstrual cycle information. In this step, user can choose Lunar Cycle if she has no period or experience irregular period.

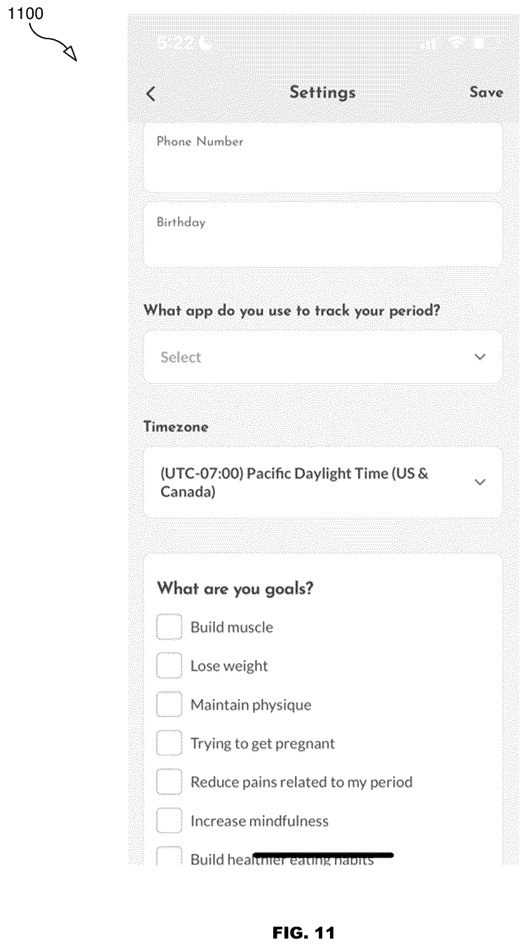

Referring to FIG. 11, screenshot 1100 shows a user choosing a time zone.

The user device is configured to transmit the inputted data to, for example, a communication network. The user data is processed in accordance with the above-described method of FIG. 5. The user device is further configured to receive menstrual cycle phase data and the feminine wellness information, and to display, on the device, the menstrual cycle phase data and the feminine wellness information. The feminine wellness information may include customized streaming workouts, nutrition profiles, recipes, and science-based “horoscope-like” insights into a user's emotions that are unique to each phase and may be unique to each day.

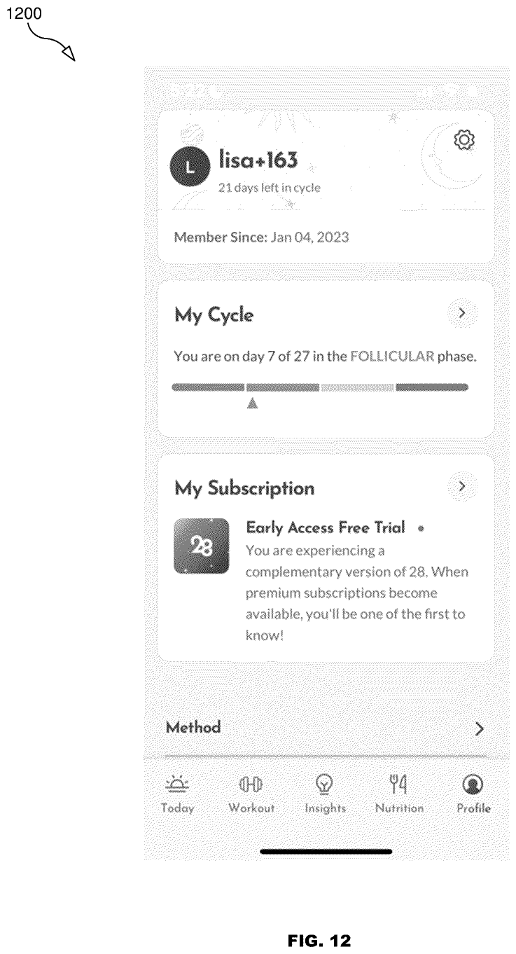

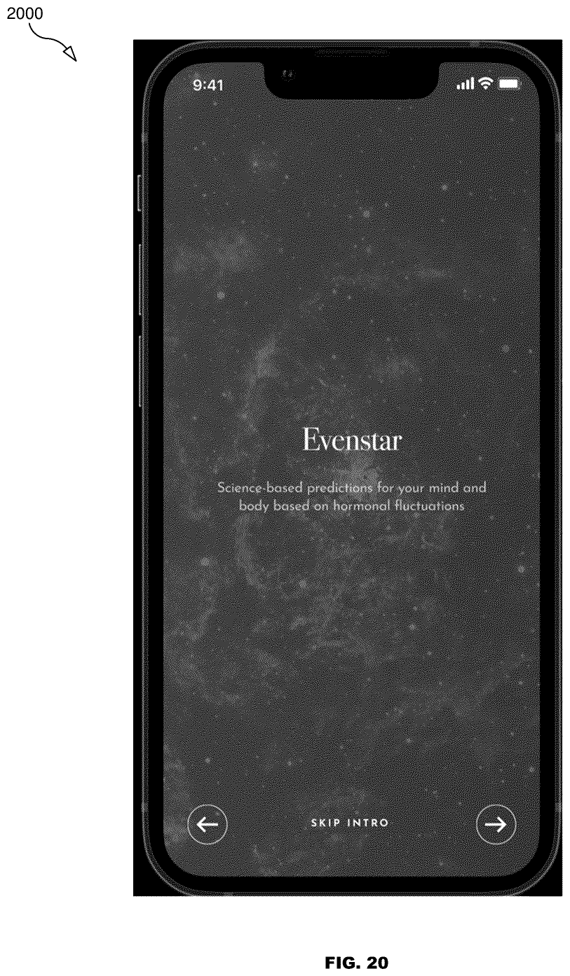

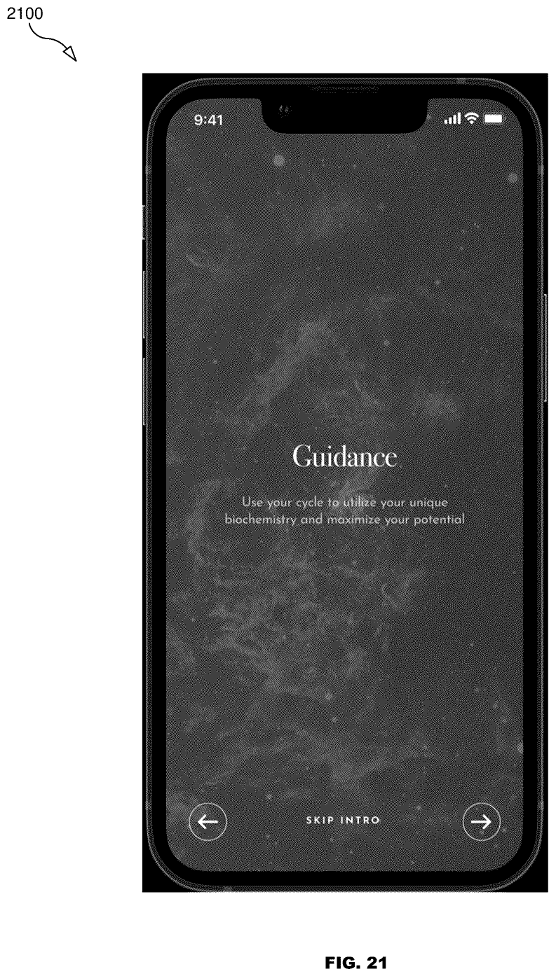

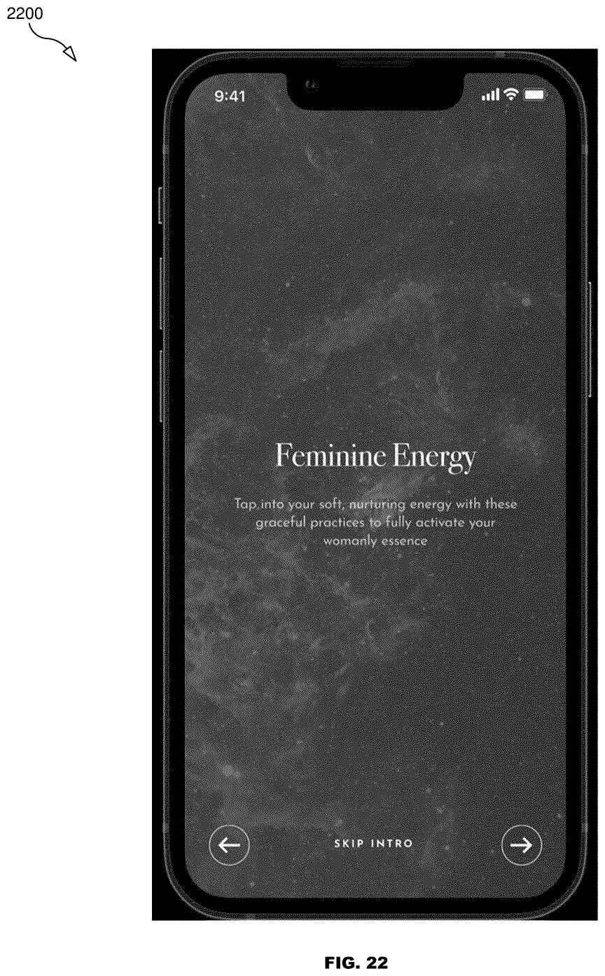

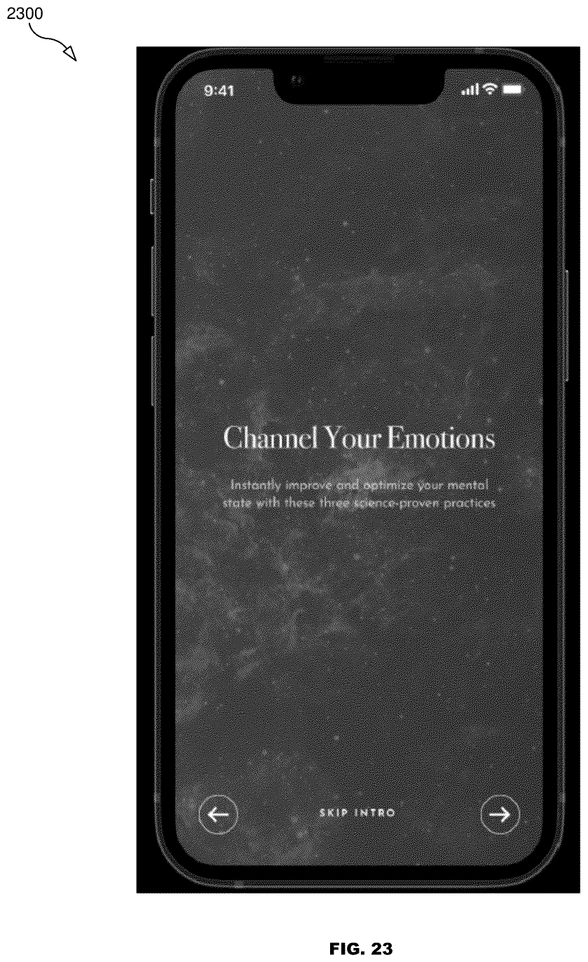

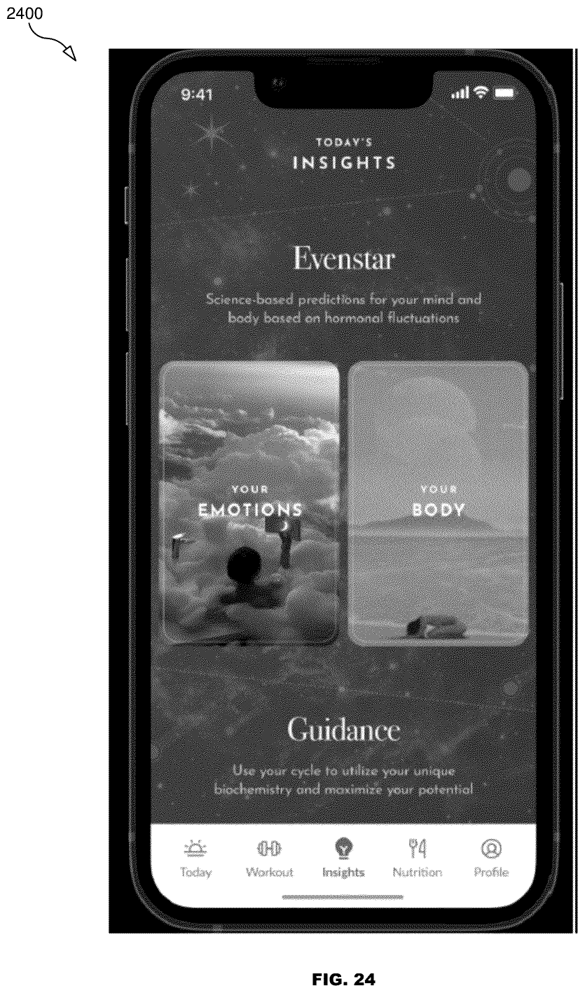

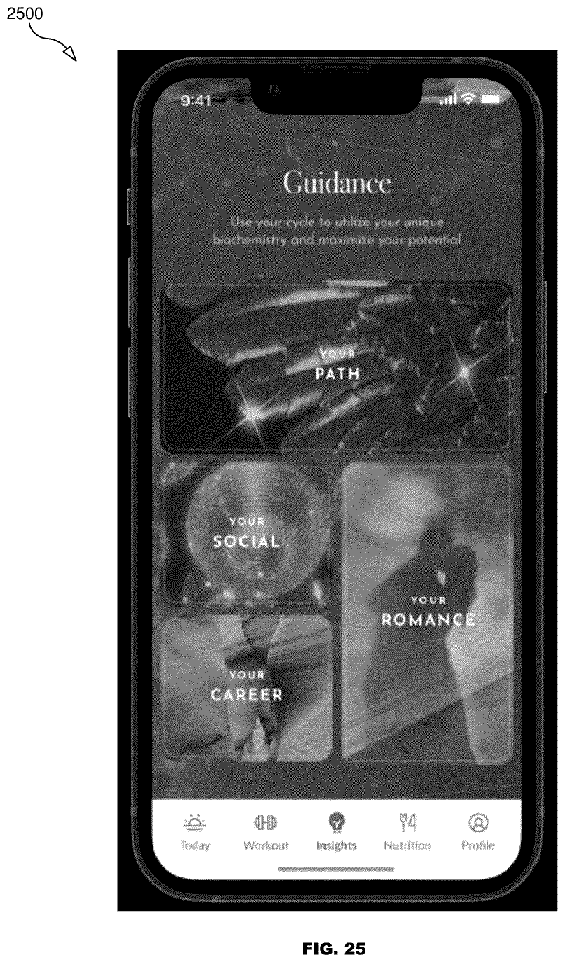

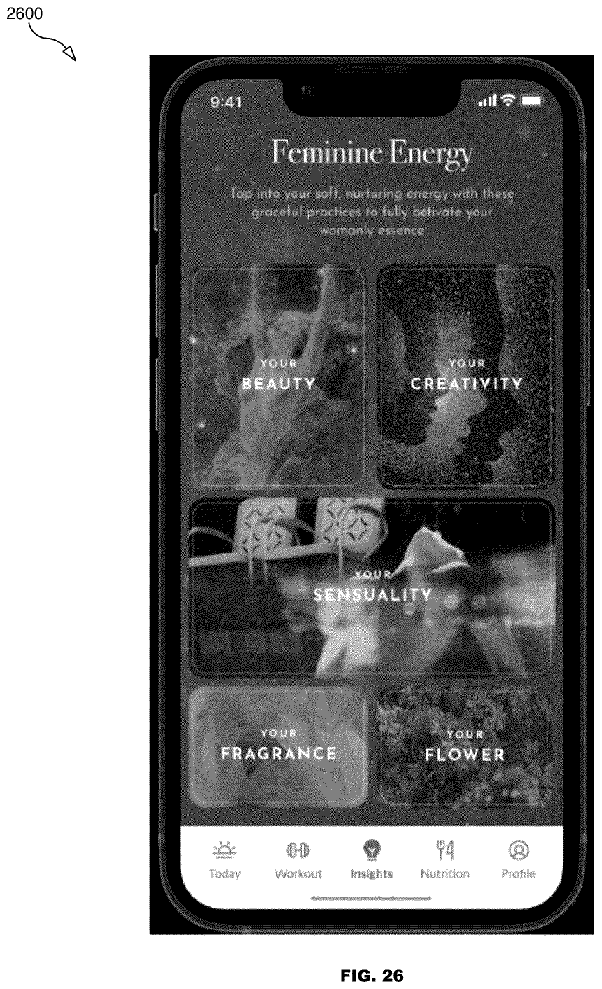

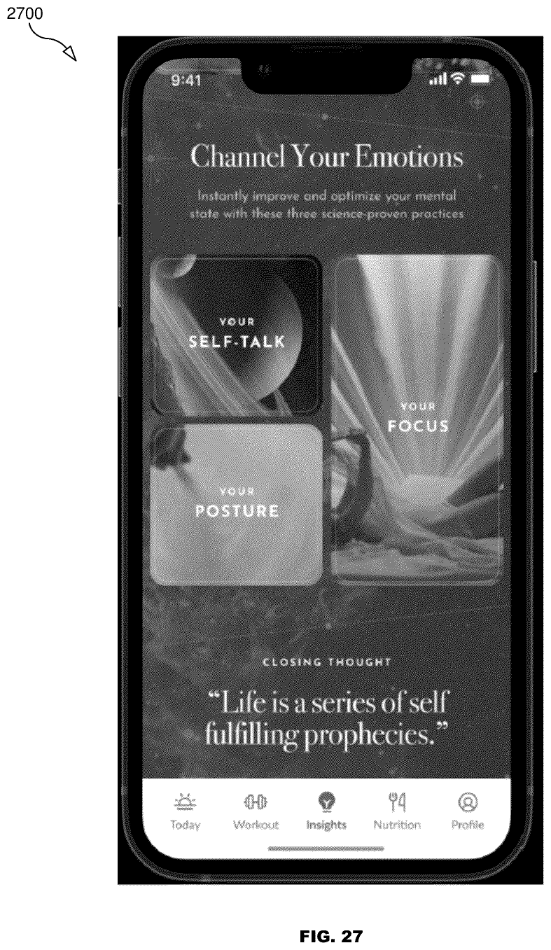

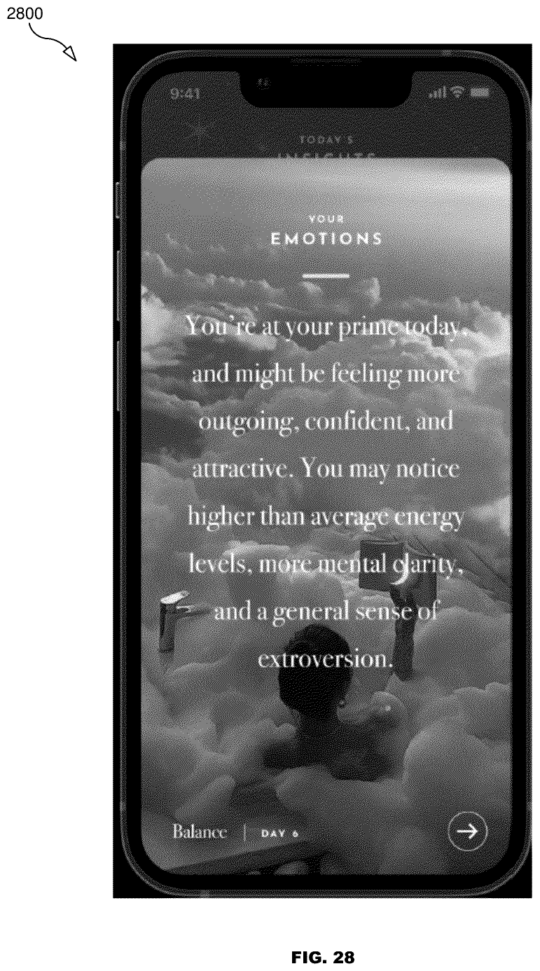

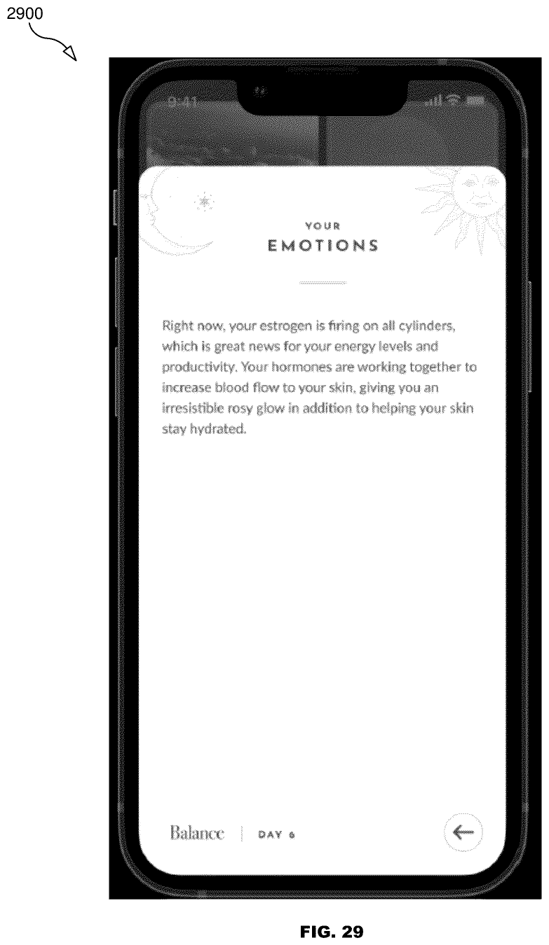

FIGS. 12-29 illustrate exemplary screenshots from an application displayed on a user device showing this functionality.

Referring to FIG. 12, screenshot 1200 shows what the application calculates the day and phase that the user is in based on the menstrual cycle information they put in. Based on the 2 pieces of information that the user puts in (first day of last cycle and average cycle duration), the application tells user that she's on what day of the cycle in which phase.

If user has cycle duration other than 28 days, the phase day will be reflected based on the calculation method described above.

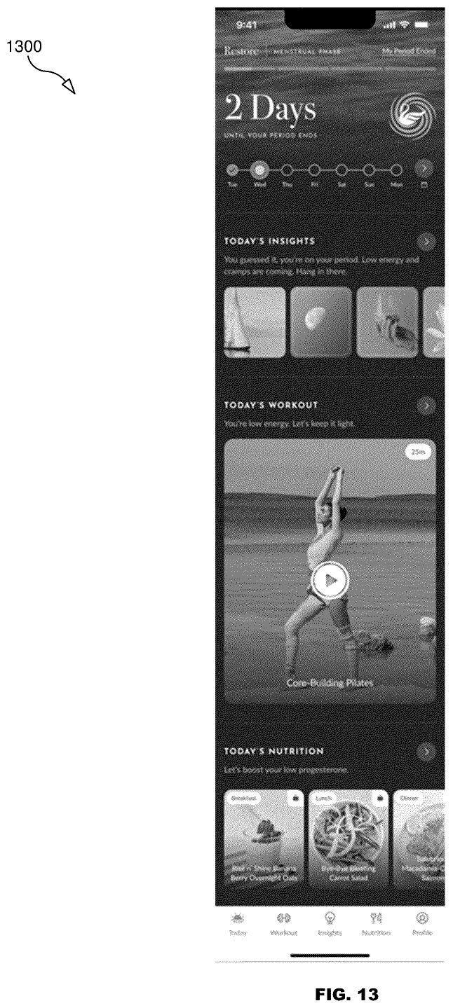

Referring to FIG. 13, screenshot 1300 shows the user what phase and day they are in.

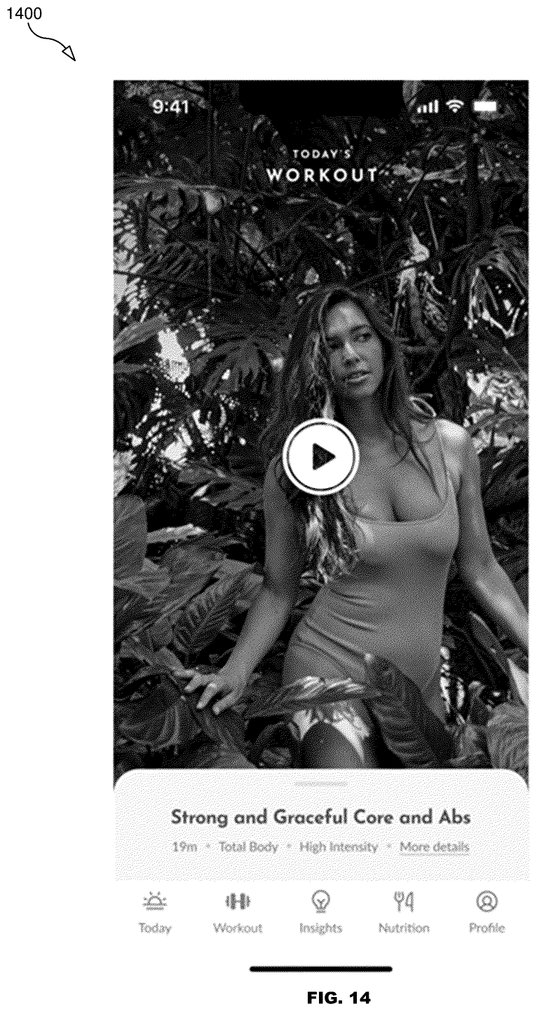

Referring to FIG. 14, screenshot 1400 shows what the application recommends for workout video content based on the phase the user is in. If user has cycle duration more than 28 days, some workout videos could be used again for multiple days. For example, “Snatched Abs” workout can be used on day 6 but also day 7 as well.

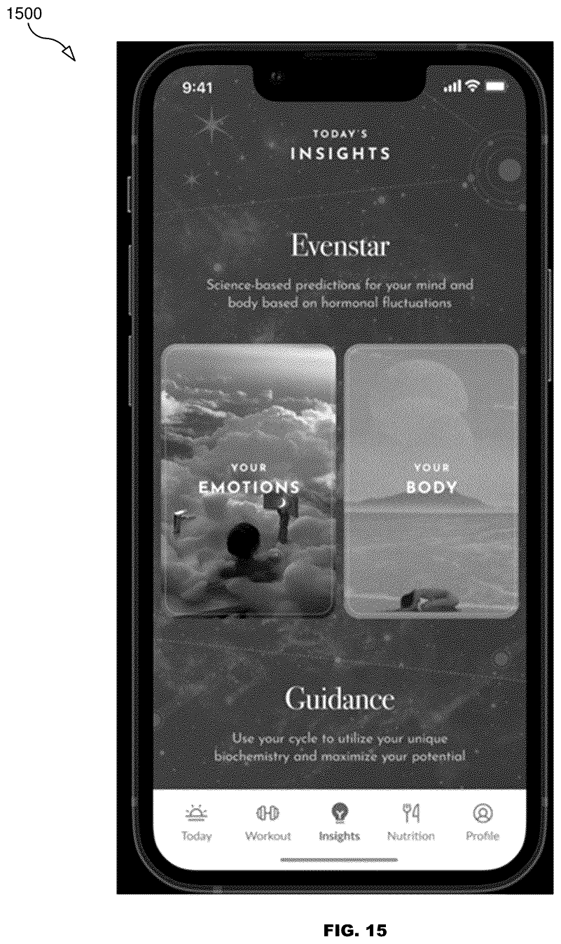

Referring to FIG. 15, screenshot 1500 shows what the application recommends for today's insights based on the phase the user is in.

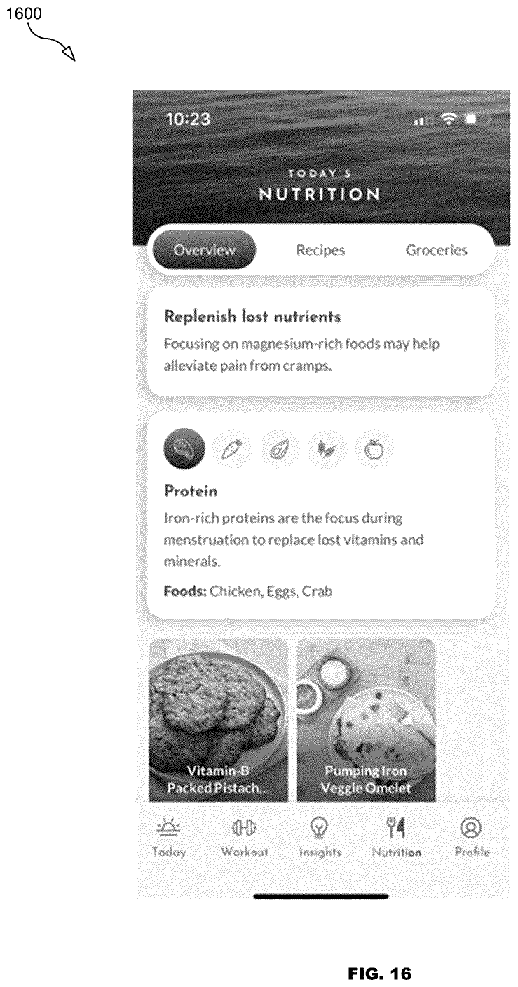

Referring to FIG. 16, screenshot 1600 shows what the application recommends for nutrition content based on the phase the user is in based on why we need to eat what food and related information.

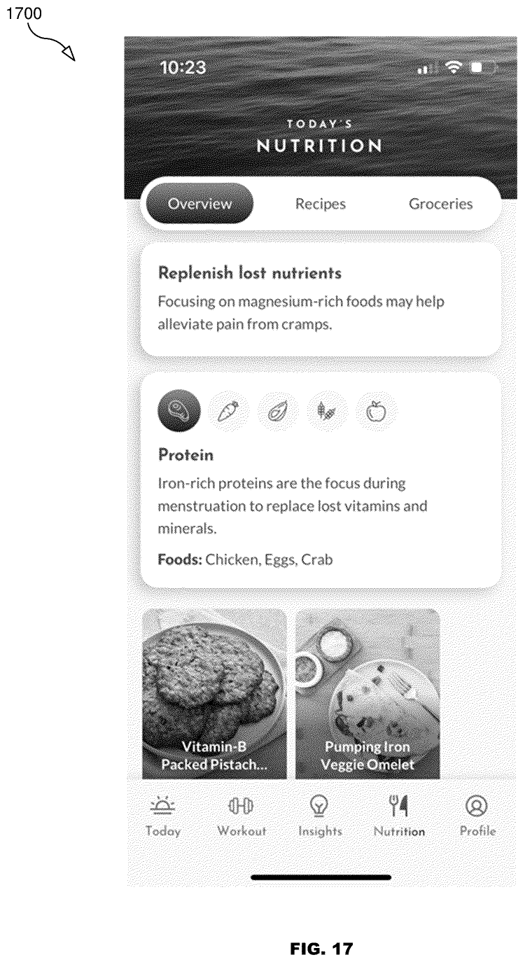

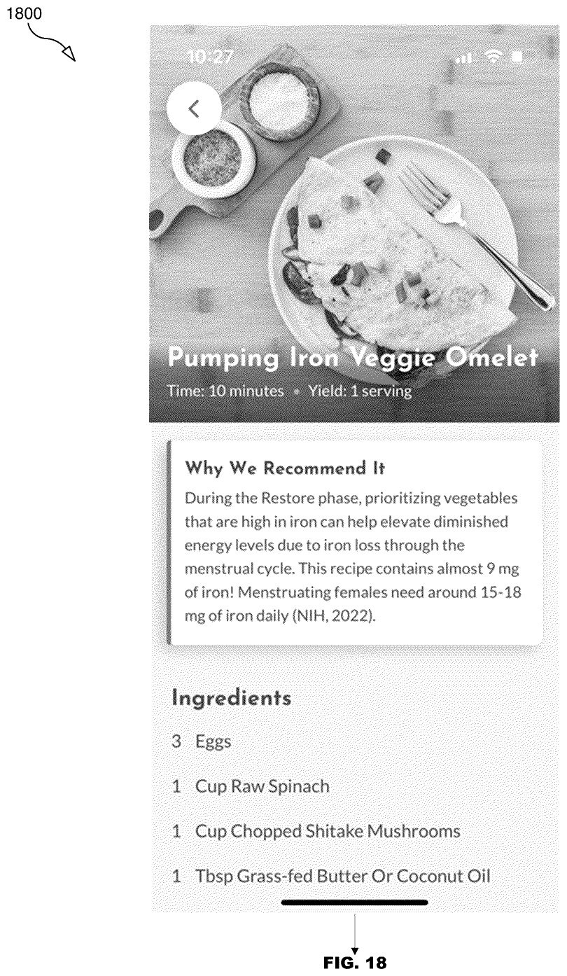

Referring to FIGS. 17 and 18, screenshots 1700 and 1800 show what the application recommends for nutrition, including nutrition recipes, and the recommendation logic is the same as how the application recommends workouts and foods based on the original nutrition content. For example, if an user is in Day 1 of the cycle, we recommend eating eggs and spinach during Restore (Menstrual) phase, so one of the recipes below is high in iron which helps elevate diminished energy level due to iron loss through the menstrual cycle

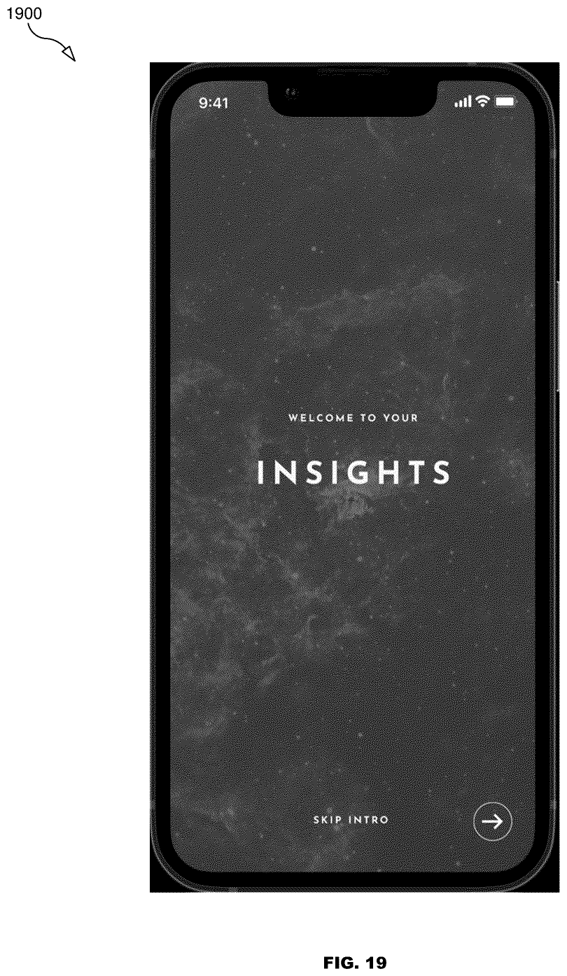

Referring to FIGS. 19-29, screenshots 1900-2900 show science based predictions for user's mind and body based on hormonal fluctuations, referred to as “Insights” and “Evenstar.” The logic of what content to be shown to users is the same as the workout videos recommendation and nutrition content.

In another aspect of the disclosed embodiments, a method is implemented in a user device 105 operating in conjunction with a wearable device 135. The information collected from the various sensors and other input mechanisms and functionalities available in the wearable device 135 can be used alone and/or in conjunction with information input by the user and/or available from the user device 105 as part of carrying out the methods of the exemplary embodiments described herein. For example, in exemplary embodiments, the following data can be obtained or collected and used as part of and/or in determining menstrual cycle phase data and/or in determining feminine wellness information to be transmitted to the user device 105.

Body/Skin Temperature:
Data to collect: Basal Body Temperature (BBT) is the temperature of the body at rest, and it slightly increases during ovulation.
Collection method: Use the temperature sensors 161 available in the wearable device 135 to record temperature regularly, preferably during sleep for consistent readings.

Heart Rate:
Data to collect: Heart rate variations during the menstrual cycle can offer insights into the different phases,
Collection method: Utilize the heart rate sensors 163 capabilities of the wearable device 135 to track changes throughout the cycle,

Physical Activity and Sleep Patterns:
Data to collect: Changes in physical activity levels and sleep patterns can indicate different phases of the menstrual cycle.
Collection method: Gather data from the position/motion sensors 160 of the wearable device 135 to monitor activity and sleep.

Hormone Levels:
Data to collect: Hormone levels such as estrogen and progesterone can provide more precise insights into the menstrual cycle.
Collection method: Integrate the wearable device 135 with other health monitoring devices and/or applications that can provide hormone level data.

User Input and Symptoms:
Data to collect: Users to manually input additional data, such as menstrual symptoms, mood, and any other relevant factors.
Collection method: Screen 206 and/or buttons/user interface 170 provide users, using the application, to input this data.
In an exemplary embodiment, the user device 105 and the wearable device 135 are configured to communicate by sending and receiving the data and other information that may have been collected, as described. The connection may be wireless, such as by using Bluetooth, and the data and other information collected may be ingested by the application running in the user device 105 through API integration, for example.

Wearable devices 135 may provide valuable physiological information that can be correlated with different phases of the menstrual cycle, as described below.
Collect and Analyze Data: Start by collecting data (through API integration) from the wearable device 135, such as body temperature, skin temperature, heart rate, and any other relevant metrics. Organize and analyze this data to understand patterns and trends over the user's menstrual cycle.
Correlate Data with Menstrual Cycle Phases: Use the data collected to identify patterns that align with different phases of the menstrual cycle (e.g., ovulatory, luteal, menstrual). For example, certain metrics may consistently vary in specific phases.
Establish Baselines: Determine baseline values for each metric during different phases of the cycle. This will help define ranges or thresholds that indicate which phase the user is likely in based on theft wearable device data.
Create Machine Learning Models: Utilize machine learning algorithms to build predictive models based on the data from wearable devices. These models can analyze the physiological data and accurately predict the current phase and phase day of the user's menstrual cycle.
User Input and Calibration: Allow users to input theft menstrual cycle data (e.g., start date of the last period) to calibrate the predictions initially. This can help the algorithm become more personalized for each user over time.

Phase Identification:
Algorithms or rules can be used to identity different phases of the menstrual cycle based on collected the data and information—i.e., metrics—collected from wearable devices 135. Common phases for which the data and information collected can be used for identification include:
   Menstruation (Day 1-5): Identify this phase based on menstrual flow data or BBT patterns.
   Follicular Phase (Day 6-14): Use changes in BBT and/or hormonal fluctuations (if available) to identify the follicular phase.
   Ovulation Phase (Day 12-16): Predict ovulation based on the BBT rise, heart rate variability (HRV) changes, and possibly hormonal data (if available).
   Luteal Phase (Day 15-28): Use BBT patterns and other metrics to identify the luteal phase.
Ovulation Phase Prediction:
   Apply machine learning algorithms for predictive models using features like BBT, HRV, and other relevant metrics to forecast the ovulation phase.
   Consider using classification techniques to determine whether a user is approaching the ovulation phase or has already ovulated.
Cycle Length Prediction:
   Use historical data and user inputs to predict the length of the next menstrual cycle.
   Machine learning regression models can be helpful for predicting cycle length.

For Predicting if a User is Ovulating:
Basal Body Temperature (BBT) Tracking: Through the application or by instructing the user, track the user's BBT every morning immediately upon waking up, before getting out of bed or engaging in any physical activity. This information can be collected through the wearable device 135.
Charting the BBT: The information collected on the daily BBT for a user can be analyzed, for example, on a graph or a digital chart. The x-axis represents the cycle days, and the y-axis shows the temperature values. Over the course of the cycle, patterns may emerge that can help identify the ovulation phase.
Identifying Pre-Ovulation Phase (Follicular Phase): In the first half of the menstrual cycle, (before ovulation), the BBT tends to be relatively lower and steadier. This phase is known as the follicular phase.
Detecting Ovulation Phase: As ovulation approaches, there is a slight drop in BBT, followed by a noticeable, increase in temperature after ovulation. This increase in BBT is due to the release of progesterone, which occurs after ovulation.
Confirming Ovulation: Ovulation is confirmed when there is a sustained temperature rise of at least 0.2-0.5 degrees Fahrenheit (0.1-0.3 degrees Celsius) that lasts for several days (usually 10-16 days) until the next menstrual period starts.

Your body temperature naturally changes a tiny bit throughout your menstrual cycle. its lower in the first part of your cycle, and then rises when you ovulate. For most people, 96°-98° Fahrenheit is theft typical temperature before ovulation. After you ovulate, it goes up to 97°−99° F.—about four-tenths of one degree higher than your usual temperature.
Menstrual Phase:
   The menstrual phase is the beginning of the menstrual cycle when bleeding occurs due to the shedding of the uterine lining.
   During this phase, basal body temperature (BBT) may initially be low but often increases slightly during the bleeding days. However, some women may experience lower temperatures during menstruation.
Follicular Phase:
   The follicular phase occurs after the menstrual phase and before ovulation.
   BBT in the follicular phase typically remains relatively stable and lower than in the luteal phase.
   There may be slight fluctuations, but there is no sustained increase in temperature yet.
Ovulation Phase:
   Ovulation usually occurs in the middle of the menstrual cycle when an egg is released from the ovary.
   BBT may dip slightly just before ovulation, and then there is a significant increase after ovulation.
   The increase in BBT is due to the release of the hormone progesterone, which occurs after ovulation.
Luteal Phase:
   The luteal phase occurs after ovulation and before the start of the next menstrual period.
   BBT remains elevated during the luteal phase due to increased progesterone levels.
   The luteal phase usually lasts for about 10-16 days until the next menstrual period begins,

In some embodiments, artificial intelligence (AI), including, for example, machine learning (ML) and generative AI (GAI) may be used to provide a more holistic cycle syncing experience to users. AI may be used to generate personalized content based on user's input, better predict future cycles and offering educational advice to users. For example, in some embodiments, the system, application, and/or functionality may include: AI generated doctor or insights chatbot, AI generated personalized workouts, and AI predicted future cycle.

In some embodiments, an AI generated chatbot may be used—e.g., Doc chatbot. The system or application will have added to it a chatbot widget or section in the app where users can ask questions about their cycle, why they are having those symptoms, why they crave for carbs etc., which is similar to asking a question to a doctor or researching all these on Google. In some embodiments, GPT3.5/4 will be integrated into the app by using API key from Open AI, which may be implemented as follows:
Gather a dataset of cycle-related questions and corresponding responses. This dataset can be used to fine-tune the chatbot's responses if necessary.
Annotate the dataset with appropriate instructions and expected outputs. For example, you might provide prompts like “I'm feeling less energy during the luteal phase, what could be the reason?” or “Suggest foods to reduce cramps during my menstrual cycle,”
Integrate the GPT-3.5 API into our app's backend using your preferred programming language (Python is commonly used for this purpose).
Use the OpenAI API to send prompts from users to the GPT-3.5 model and receive responses. Structure the API calls to pass in the user's question as the prompt.
Craft prompts that are clear and instructive. Include specific details about the user's query, and you can also provide a persona context for the chatbot (e.g., “I am a wellness expert”).
Ask the model to think step-by-step or provide detailed explanations in its responses, depending on the complexity of the question.
Receive the model's response from the API call.
Process and sanitize the response to remove any potentially inappropriate or irrelevant content.
If you're using a fine-tuned model, you might compare the generated response with the expected response from your dataset to ensure accuracy.
Continuously gather user feedback on the chatbot's responses.
Fine-tune the prompts and instructions based on user interactions and correct the model's responses as needed to improve accuracy and relevance.

The following are examples of the implementation of this AI generated “Doc” chatbot:

### Prompt Example

Prompt
You are Dr. Andre, a well-renown doctor who is extremely knowledgable of women's health. You coach women and give them insight to match lifestyle choices and optimizations they can make with the phase they are in their cycle. I will start a conversation now and you respond in a concise, knowledgable, yet very conversational friendly style.

Response
Of course! I'm here to help. What can I assist you with regarding your cycle or any other women's health concerns?

Prompt
My period finished 3 days ago, but I'm spotting today, can you tell why and how I might be able to mitigate this?

Response:
Certainly! Spotting after a period is not uncommon, and it's often not something to be overly concerned about. Here's a bit of insight:
Residual Blood: Sometimes, a little bit of menstrual blood remains in the uterus after your period ends, and it may take a few days to exit the body. This is typically normal.
Hormonal Fluctuations: Changes in hormones can lead to spotting. If you're on birth control or recently changed birth control methods, this could be a contributing factor.
Infections or Other Medical Conditions: In rare cases, spotting can be a sign of an underlying health issue, such as an infection, polyps, or even fibroids. To mitigate spotting, maintaining a balanced lifestyle can help. Ensuring you have a nutrient-rich diet, regular exercise, and stress management can all contribute to a more stable menstrual cycle.
However, if this spotting becomes a consistent issue or if you experience other symptoms, it would be wise to consult with a healthcare provider, as an examination might be necessary to rule out any underlying issues.

In some embodiments, an AI generated chatbot may be used for insights—e.g., Insights chatbot. The Insights chatbot is similar to the doctor bot above but for Evenstar (Insights) to provide insights to users regarding their emotions, body etc. Here, stricter guidelines would be applied to the OpenAI API in order to keep responses aligned to the specific Insight that a user is asking about. If a user is viewing a Romance Insight, they could only ask and get responses that are relevant to that insight.

In some embodiments, AI can be used to generate personalized interactive content including one or more of personalized fitness routines, exercise routines, and/or video workouts. In some embodiments, this functionality can be implemented as follows:
Exercise Move Library:
   We will film a variety of exercise moves that cover different muscle groups and fitness levels (e.g. butt lift, russian twist, downward dog) Metadata Tagging;
   Tag each exercise move with relevant metadata, such as intensity level (low impact, high impact), muscle groups targeted, equipment needed, and any other relevant attributes.
User Input Processing:
   When a user inputs their preferences (e.g., “I want a 20-minute low impact gentle workout”), parse theft input to extract relevant information, such as duration and intensity level,
AI Algorithm Integration:
   Utilize AI algorithms, to generate a workout routine based on user preferences.
   Provide the AI that we're using with what our Exercise Move Library contains with specifics down to the length of a move and how many reps each move includes.
   Structure a prompt that includes the user's input and asks the AI to generate a sequence of exercise moves. For example, “Create a 20-minute low impact workout routine including exercises that target different muscle groups,”
Exercise Selection:
   Based on the AI-generated response, select exercise moves from your library that align with the generated workout routine. We'll use the metadata tags to filter and select appropriate exercises.
Video Compilation:
   For each selected exercise move, retrieve the corresponding video clip from your library,
   Use video editing software or libraries to stitch together the selected video cups into a coherent workout video and then display it to the user in app near real time:
      Use a video editing library or software that allows programmatic video editing and manipulation. Libraries Ike FFmpeg or video editing frameworks within programming languages like Python can be very useful for this purpose.
      Create a script that takes the selected video clips and dynamically compiles them into a single video. This script should handle video concatenation, transitions, overlays, and other effects.
      Store all of the individual workout videos on a user's device and pass down only the necessary metadata from our API to the client devices that would avow for client devices to potentially create a seamless playlist-esque workout video using loops and video stitches with a specific audio overlay,
In some embodiments, AI can be used to predict future cycles. AI may be used to generated chatbot may be used for personalized workouts. AI can be used to backfill data, and predict the user's future cycle. In some embodiments, the system or application will be integrated with Open AI, similar to the implementations described above, using 3 user attributes: avg_cycle_length, period_duration, date_of_last_period (which are collected by the app and stored in the database), and use Open AI to predict future cycles date, duration as well as

```
      Prompt:
Here's some historical data from a woman's last few period cycles with
‘period_start‘ and ‘period_end‘. Could you help calculate and predict:
- average cycle length
- menstral duration
- next period prediction
input rows for last 3 historical periods in triple backticks
‘‘‘
{
{“period_start”: “2023/07/23”, “period_end”: “2023/07/27”},
{“period_start”: “2023/06/29”, “period_end”: “2023/07/04”},
{“period_start”: “2023/06/04”, “period_end”: “2023/06/08”},
}
‘‘‘
Can you provide the output in a JSON format with attributes: ‘avg_cycle_len‘,
‘menstral_duration‘, ‘date_of_next_period_prediction‘. I do not need an
explanation or code example, just the concise output.
Response:
{
  “avg_cycle_len”: 25.0,
  “menstral_duration”: 4.0,
  “date_of_next_period_prediction”: “2023-08-17”
}
```

The computer-implemented method may be executed using Mux (Video), MySQL/Vitess/Planetscale (Database) Vercel (Serverless deployments+edge functions), Auth0 (Authentication/Authorization), Segment (Analytics), Sentry (Error & Exception Tracking), Stripe (Payments).

Open source software to implement the method may include React & Next.js (UI Library & Framework respectively) and Playwright (End to end testing).

Additional subject matter is included in the attached Appendix which is part of this disclosure.

### EMBODIMENTS

A1. A computer-implemented method for providing feminine wellness information to one or more users, the method comprising:
receiving, from a device, menstrual cycle data from a user, wherein the menstrual cycle data includes a first date of a last menstrual cycle;
determining menstrual cycle phase data using the menstrual cycle data;
determining feminine wellness information using menstrual cycle phase data; and
transmitting, to the device, the menstrual cycle phase data and the feminine wellness information, wherein the feminine wellness information comprises a fitness routine recommendation.
A2. The method of embodiment A1, wherein the feminine wellness information further comprises nutrition profiles.
A3. The method of embodiment A1, wherein the feminine wellness information further comprises physiological data.
A4. The method of embodiment A1, wherein the fitness routine recommendation is a video of a fitness routine.
A5. The method of embodiment A4 further comprising selecting the video from the dashboard and streaming the video on the display screen.
A6. The method of embodiment A1, wherein menstrual cycle data includes a cycle duration.
B1. A computer-implemented method for providing feminine wellness information to one or more users, the method comprising:
receiving, from a device, menstrual cycle data from a user, wherein the menstrual cycle data includes a first date of a last menstrual cycle;
receiving, from a device, a first current date;
calculating a first number of days since the first date of the last menstrual cycle using the first current date and the menstrual cycle data;
determining a first menstrual cycle phase data using the first number of days since the first date of the last menstrual cycle;
determining a first feminine wellness information using the first menstrual cycle phase data; and
transmitting, to the device, the first menstrual cycle phase data and the first feminine wellness information, wherein the feminine wellness information comprises a first fitness routine recommendation.
B2. The method of embodiment B1 further comprising:
receiving, from the device, a second current date;
calculating a second number of days since the first date of the last menstrual cycle using the second current date and the menstrual cycle data;
determining a second menstrual cycle phase data using the second number of days since the second date of the last menstrual cycle;
determining a second feminine wellness information using the second menstrual cycle phase data; and
transmitting, to the device, the second menstrual cycle phase data and the second feminine wellness information, wherein the feminine wellness information comprises a second fitness routine recommendation.
B3. The method of embodiment B1, wherein menstrual cycle data includes a cycle duration.
B4. The method of embodiment B1, wherein determining menstrual cycle phase data includes converting menstrual cycle data to Coordinated Universal Time (UTC).
B5. The method of embodiment B3, wherein determining the first menstrual cycle phase data includes calculating a day that the user is in their cycle using the first number of days since the first date of the last menstrual cycle and the cycle duration.
B6. The method of embodiment B5, wherein determining the first menstrual cycle phase data further includes converting the day that the user is in their cycle to a day within a phase day range using the user's cycle duration and a maximum of the phase day range.
B7. The method of embodiment B6, wherein the phase day range is 1-28 and the maximum of the phase day range is 28.
C1. A computer-implemented method for providing feminine wellness information to one or more users, the method comprising:
inputting, in a device, menstrual cycle data, wherein the menstrual cycle data includes a first date of a last menstrual cycle;
transmitting, from the device, the inputted menstrual cycle data;
receiving, in the device, the menstrual cycle phase data and the feminine wellness information; and
displaying, on the device, the received menstrual cycle phase data and the feminine wellness information, wherein the feminine wellness information comprises a fitness routine recommendation.
C2. The method of embodiment C1 further comprising:
receiving, in the device, a first menstrual cycle phase data and a first feminine wellness information; and
displaying, on the device, the first menstrual cycle phase data and the first feminine wellness information, wherein the feminine wellness information comprises a first fitness routine recommendation.
C3. The method of embodiment C2 further comprising:
receiving, in the device, a second menstrual cycle phase data and a second feminine wellness information; and
displaying, on the device, the second menstrual cycle phase data and the second feminine wellness information, wherein the feminine wellness information comprises a second fitness routine recommendation.
C4. The method of embodiment C1, wherein menstrual cycle data includes a cycle duration.
D1. A device or node adapted to perform the method of any one of embodiments A1 to A5, B1 to B2, and C1 to C3.
E1. A computer program comprising instructions which when executed by processing circuitry of a device or node causes the device or node to perform the method of any one of embodiments A1 to A5, B1 to B2, and C1 to C3.
F1. A carrier containing the computer program of embodiment E1, wherein the carrier is one of an electronic signal, an optical signal, a radio signal, and a computer readable storage medium.

1. A computer-implemented method for providing feminine wellness information to one or more users, the method comprising:
receiving, from a device, menstrual cycle data for a user, wherein the menstrual cycle data includes one or more of a first date of the user's last menstrual cycle, a cycle length of the user's menstrual cycle, and/or a length of the user's period;
determining menstrual cycle phase data using the menstrual cycle data;
determining feminine wellness information using the menstrual cycle phase data, wherein the feminine wellness information includes personalized interactive content; and
transmitting, to the device, the menstrual cycle phase data and the feminine wellness information including the personalized interactive content.

2. The method according to claim 1, wherein the feminine wellness information includes one or more of a fitness routine recommendation, a nutrition profile, a recipe, and/or physiological data.

3. The method according to claim 1, wherein determining feminine wellness information using the menstrual cycle phase data includes using a cycle syncing content recommendation engine algorithm.

4. The method according to claim 3, wherein:
inputs to the cycle syncing content recommendation engine algorithm include one or more of a start date of the user's menstrual cycle, a cycle length of the user's menstrual cycle, and/or a length of the user's period; and
outputs from the cycle syncing content recommendation engine algorithm include one or more of curated content targeted specifically to where the user is in the user's menstrual cycle.

5. The method according to claim 1, wherein determining menstrual cycle phase data using the menstrual cycle data includes using information collected from a wearable device worn by the user, wherein the collected information includes one or more of the user's body and/or skin temperature, heart rate, physical activity, sleep patterns, hormone levels, menstrual symptoms, and/or mood.

6. The method according to claim 1, wherein determining feminine wellness information using the menstrual cycle phase data includes using an artificial intelligence (AI)/machine learning (ML) model for providing cycle syncing content recommendations.

7. The method according to claim 1, wherein determining feminine wellness information using the menstrual cycle phase data includes using a generative artificial intelligence (GAI) module to generate personalized feminine wellness information.

8. The method according to claim 7, wherein the GAI generated personalized feminine wellness information includes personalized interactive content including one or more of personalized fitness routines, exercise routines, and/or video workouts.

9. A computer-implemented method for providing feminine wellness information to one or more users, the method comprising:
receiving, from a device, menstrual cycle data for a user, wherein the menstrual cycle data includes a first date of a last menstrual cycle;
receiving, from a device, a first current date;
calculating a first number of days since the first date of the last menstrual cycle using the first current date and the menstrual cycle data;
determining a first menstrual cycle phase data using the first number of days since the first date of the last menstrual cycle;
determining a first feminine wellness information using the first menstrual cycle phase data, wherein the first feminine wellness information includes first personalized interactive content; and
transmitting, to the device, the first menstrual cycle phase data and the first feminine wellness information including the first personalized interactive content.

10. The method according to claim 9 further comprising:
receiving, from the device, a second current date;
calculating a second number of days since the first date of the last menstrual cycle using the second current date and the menstrual cycle data;
determining a second menstrual cycle phase data using the second number of days since the second date of the last menstrual cycle;
determining a second feminine wellness information using the second menstrual cycle phase data, wherein the second feminine wellness information includes second personalized interactive content; and
transmitting, to the device, the second menstrual cycle phase data and the second feminine wellness information including the second personalized interactive content.

11. The method according to claim 9, wherein menstrual cycle data includes a cycle duration and determining the first menstrual cycle phase data includes calculating a day that the user is in their cycle using the first number of days since the first date of the last menstrual cycle and the cycle duration.

12. The method according to claim 11, wherein determining the first menstrual cycle phase data further includes converting the day that the user is in their cycle to a day within a phase day range using the user's cycle duration and a maximum of the phase day range.

13. The method according to claim 12, wherein the phase day range is 1-28 and the maximum of the phase day range is 28.

14. A computer-implemented method for receiving feminine wellness information for a user, the method comprising:
inputting, in a device, menstrual cycle data, wherein the menstrual cycle data includes one or more of a first date of the user's last menstrual cycle, a cycle length of the user's menstrual cycle, and/or a length of the user's period;
transmitting, from the device, the inputted menstrual cycle data;
receiving, in the device, the menstrual cycle phase data and the feminine wellness information including personalized interactive content; and
displaying, on the device, the received menstrual cycle phase data and the feminine wellness information including the personalized interactive content.

15. The method according to claim 14, wherein the feminine wellness information includes one or more of a fitness routine recommendation, a nutrition profile, a recipe, and/or physiological data.

16. The method according to claim 14, wherein the menstrual cycle data includes information collected from a wearable device worn by the user, wherein the collected information includes one or more of the user's body and/or skin temperature, heart rate, physical activity, sleep patterns, hormone levels, menstrual symptoms, and/or mood.

17. The method according to claim 14, wherein determining feminine wellness information using the menstrual cycle phase data includes using a generative artificial intelligence (GAI) module to generate personalized feminine wellness information.

18. The method according to claim 14, wherein the feminine wellness information received includes GAI generated personalized feminine wellness information including one or more of personalized fitness routines, exercise routines, and/or video workouts.

19. A device for providing feminine wellness information to one or more users, comprising a processing circuitry and a memory storing program code that, when executed by the processing circuitry, causes the device to carry out the method according to claim 1.

20. A device for receiving feminine wellness information for a user, comprising a processing circuitry and a memory storing program code that, when executed by the processing circuitry, causes the device to carry out the method according to claim 14.

21. A computer program product comprising a non-transitory computer readable medium storing a computer program comprising instructions which, when executed on at least one processor, cause the at least one processor to carry out the method according to claim 1.

22. A computer program product comprising a non-transitory computer readable medium storing a computer program comprising instructions which, when executed on at least one processor, cause the at least one processor to carry out the method according to claim 14.
